(19) United States
(12) Patent Application Publication
(10) Pub. No.: US 2024/0261282 A1
(43) Pub. Date: Aug. 8, 2024

(54) PHARMACEUTICAL COMPOSITIONS AND COMBINATIONS COMPRISING INHIBITORS OF THE ANDROGEN RECEPTOR AND USES THEREOF

(71) Applicant: ESSA Pharma Inc., Vancouver (CA)

(72) Inventors: Peter VIRSIK, Portola Valley, CA (US); Han-Jie ZHOU, Foster City, CA (US)

(21) Appl. No.: 18/595,842

(22) Filed: Mar. 5, 2024

Related U.S. Application Data
(62) Division of application No. 17/476,904, filed on Sep. 16, 2021.
(60) Provisional application No. 63/078,987, filed on Sep. 16, 2020.

(51) Int. Cl. A61K 31/505; A61K 31/415; A61K 31/4164; A61K 31/4439; A61K 31/573; A61K 31/58; A61K 47/38; A61P 35/00

(52) U.S. Cl. CPC A61K 31/505; A61K 31/415; A61K 31/4164; A61K 31/4439; A61K 31/573; A61K 31/58; A61K 47/38; A61P 35/00

(57) ABSTRACT

The present disclosure generally relates to pharmaceutical compositions and combinations comprising N-(4-((4-(2-(3-chloro-4-(2-chloroethoxy)-5-cyanophenyl)propan-2-yl)phenoxy)methyl)pyrimidin-2-yl)methanesulfonamide or a pharmaceutically acceptable salt, solvate, stereoisomer, or prodrug thereof, and a second therapeutically active agent, such as an antiandrogen. In particular, the present disclosure relates to pharmaceutical compositions and combinations useful for treatment of various cancers, for example breast cancer and prostate cancer.

### FIELD OF THE INVENTION

This application is a division of U.S. application Ser. No. 17/476,904, filed Sep. 16, 2021, which claims priority to U.S. Provisional Application No. 63/078,987, filed Sep. 16, 2020, the disclosures of each of which are hereby incorporated by reference in their entireties for all purposes.

### FIELD OF THE INVENTION

The present disclosure generally relates to pharmaceutical compositions and combinations comprising an androgen receptor (AR)N-terminal domain inhibitor (NTD) Compound A and an additional therapeutic agent, such as an antiandrogen. In particular, the present disclosure relates to pharmaceutical compositions and combinations useful for treatment of various cancers, such as prostate cancer.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration also known as androgen ablation therapy (ABT) or androgen deprivation therapy (ADT).

Androgen receptor (AR) is a transcription factor that plays dual roles in breast cancer cells: promoting or inhibiting proliferation depending on expression and activity of estrogen receptor-alpha. Currently there is no FDA-approved targeted therapy for triple-negative breast cancer (TNBC). AR plays a role in the proliferation of breast cancer cells by either promoting proliferation or inhibiting proliferation depending on the expression of estrogen receptor (ER) and human epidermal growth factor receptor 2 (HER2). AR expression is detected in up to 90% of all breast cancers and in up to approximately 35% of TNBC. AR-Vs have been detected in primary breast cancer specimens and in breast cancer cell lines. AR-V7 expression was detected in circulating-tumor cells of patients with metastatic breast cancer and was associated with bone metastases. Targeting AR is a potential therapeutic strategy for AR-positive TNBC.

Androgens also play a role in female diseases such as polycystic ovary syndrome as well as cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate luminal cells. Androgen ablation therapy causes a temporary reduction in tumor burden concomitant with a decrease in serum prostate-specific antigen (PSA). Unfortunately, prostate cancer can eventually grow again in the absence of testicular androgens (castration-resistant disease) (Huber et al 1987 Scand J. Urol Nephrol. 104, 33-39). Castration-resistant prostate cancer that is still driven by AR is biochemically characterized before the onset of symptoms by a rising titre of serum PSA (Miller et al 1992 J. Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains two transcriptional activation units (tau1 and tau5) within activation function-1 (AF-1). Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (TL-6) and by various growth factors (Culig et al 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

Clinically available inhibitors of the AR include nonsteroidal antiandrogens such as bicalutamide (Casodex™), nilutamide (Anandron®, Nilandron®), flutamide (Eulexin®), enzalutamide (Xtandi®), apalutamide (Erleada®), and darolutamide (Nubega®). There is also a class of steroidal antiandrogens, such as cyproterone acetate and spironolactone. Both steroidal and non-steroidal antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)), and constitutively active AR splice variants. Antiandrogens have no effect on the constitutively active AR splice variants that lack the ligand-binding domain (LBD) and are associated with castration-recurrent prostate cancer (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30) and resistant to abiraterone and enzalutamide (Antonarakis et al., N Engl J Med. 2014, 371, 1028-38; Scher et al JAMA Oncol. 2016 doi: 10.1001). Conventional therapy has concentrated on androgen-dependent activation of the AR through its C-terminal domain.

Other relevant AR antagonists previously reported (see, WO 2010/000066, WO 2011/082487; WO 2011/082488; WO 2012/145330; WO 2015/031984; WO 2016/058080; and WO 2016/058082) that bind to full-length AR and/or truncated AR splice variants that are currently being developed include: AR degraders such as niclosamide (Liu C et al 2014), galeterone (Njar et al 2015; Yu Z at al 2014), and ARV-330/Androgen receptor PROTAC (Neklesa et al 2016 J Clin Oncol 34 suppl 2S; abstr 267); AR DBD inhibitor VPC-14449 (Dalal K et al 2014 J Biol Chem. 289(38):26417-29; Li H et al 2014 J Med Chem. 57(15):6458-67); antiandrogens apalutamide (Clegg N J et al 2012), ODM-201 (Moilanen A M et al 2015), ODM-204 (Kallio et al J Clin Oncol 2016 vol. 34 no. 2_suppl 230), TAS3681 (Minamiguchi et al 2015 J Clin Oncol 33, suppl 7; abstr 266); and AR NTD inhibitors 3E10-AR441bsAb (Goicochea N L et al 2015), and sintokamide (Sadar et al 2008; Banuelos et al 2016).

The AR-NTD is also a target for drug development (e.g. WO 2000/001813; Myung et al. J. Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J. Biol. Chem. 274, 7777-7783; Sadar M D et al 1999 Endocr Relat Cancer. 6, 487-502; Ueda et al 2002 J. Biol. Chem. 277, 7076-7085; Ueda 2002 J. Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches.

Compounds that modulate AR, potentially through interaction with NTD domain, include the bisphenol compounds disclosed in published PCT Nos: WO 2010/000066, WO 2011/082487; WO 2011/082488; WO 2012/145330; WO 2012/139039; WO 2012/145328; WO 2013/028572; WO 2013/028791; WO 2014/179867; WO 2015/031984; WO 2016/058080; WO 2016/058082; WO 2016/112455; WO 2016/141458; WO 2017/177307; WO 2017/210771; and WO 2018/045450, and which are hereby incorporated by reference in their entireties.

Transcriptionally active androgen receptor plays a major role in CRPC in spite of reduced blood levels of androgen (Karantanos, T. et al Oncogene 2013, 32, 5501-5511; Harris, W. P. et al Nature Clinical Practice Urology, 2009, 6, 76-85). AR mechanisms of resistance to ADT include: overexpression of AR (Visakorpi, T. et al Nature Genetics 1995, 9, 401-406; Koivisto, P. et al Scandinavian Journal of Clinical and Laboratory Investigation Supplementum 1996, 226, 57-63); gain-of-function mutations in the AR LBD (Culig Z. et al Molecular Endocrinology 1993, 7, 1541-1550); intratumoral androgen synthesis (Cai, C. et al Cancer Research 2011, 71, 6503-6513); altered expression and function of AR coactivators (Ueda, T. et al The Journal of Biological Chemistry 2002, 277, 38087-38094; Xu J. et al Nature Reviews Cancer 2009, 9, 615-630); aberrant post-translational modifications of AR (Gioeli D. et al Molecular and Cellular Endocrinology 2012, 352, 70-78; van der Steen T. et al International Journal of Molecular Sciences 2013, 14, 14833-14859); and expression of AR splice variants (AR-Vs) which lack the ligand-binding domain (LBD) (Karantanos, T. et al Oncogene 2013, 32, 5501-5511; Andersen R. J. et al Cancer Cell 2010, 17, 535-546; Myung J. K. et al The Journal of Clinical Investigation 2013, 123, 2948-2960; Sun S. et al The Journal of Clinical Investigation 2010, 120, 2715-2730). Anti-androgens such as bicalutamide and enzalutamide target AR LBD, but have no effect on truncated constitutively active AR-Vs such as AR-V7 (Li Y. et al Cancer Research 2013, 73, 483-489). Expression of AR-V7 is associated with resistance to current hormone therapies (Li Y. et al Cancer Research 2013, 73, 483-489; Antonarakis E. S. et al The New England Journal of Medicine 2014, 371, 1028-1038).

While significant advances have been made in this field, there remains a need for improved treatment for AR-mediated disorders including prostate cancer.

### SUMMARY OF THE INVENTION

The present disclosure relates to pharmaceutical compositions and combinations comprising Compound A and at least one additional therapeutically active agent. In one embodiment, the composition and/or combination comprises a second therapeutically active agent. In one embodiment, the composition and/or combination comprises a second therapeutically active agent and a third therapeutically active agent. In one embodiment, the composition and/or combination comprises a second therapeutically active agent, a third therapeutically active agent, and a fourth therapeutically active agent.

The present disclosure relates to a pharmaceutical combination comprising a therapeutically effective amount of a first therapeutically active agent Compound A or a pharmaceutically acceptable salt, solvate, stereoisomer or prodrug thereof, and a second therapeutically active agent in at least one pharmaceutical composition.

In one embodiment of the pharmaceutical combination of the present disclosure, the second therapeutically active agent is an androgen receptor ligand-binding domain inhibitor, a steroid, a CYP17 inhibitor, a CYP3A4 inhibitor, or an inhibitor of UGT enzyme.

In one embodiment of the pharmaceutical combination of the present disclosure, the androgen receptor ligand-binding domain inhibitor is enzalutamide, apalutamide, darolutamide, bicalutamide, nilutamide, flutamide, ODM-204, TAS3681, and/or a pharmaceutically acceptable salt or solvate thereof. In one embodiment, the CYP17 inhibitor is galeterone, abiraterone, abiraterone acetate, and/or a pharmaceutically acceptable salt, solvate, or prodrug thereof. In one embodiment, steroid selected from aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, mometasone, mometasone paramethasone, mometasone furoate monohydrate, prednicarbate, methylprednisolone, prednisolone, prednisone, tixocortol, tixocortol pivalate, triamcinolone, triamcinolone acetonide, or triamcinolone alcohol, or a pharmaceutically acceptable salt or solvate thereof. In one embodiment, the CYP3A4 inhibitor is clarithromycin, telithromycin, erythromycin, nefazodone, atazanavir, darunavir, indinavir, lopinavir, nelfinavir, saquinavir, tipranavir, ritonavir, ketoconazole, itraconazole, fluconazole, verapamil, cobicistat, and/or a pharmaceutically acceptable salt or solvate thereof. In one embodiment, the inhibitor of UGT enzyme is atazanavir, indinavir, lopinavir, nelfinavir, ritonavir, saquinavir, silybin, ritonavir, quinidine, diclofenac, everolimus, gemfibrozil, androsterone, phenylbutazone, ketoconazole, nilotinib, deoxyschizandrin, hecogenin, niflumic acid, efavirenz, or amitriptyline, or a pharmaceutically acceptable salt or solvate thereof.

In one embodiment of the pharmaceutical combination of the present disclosure, the combination of Compound A and the second therapeutically active agent is in a single fixed dosage form. In one embodiment, the combination of Compound A and the second therapeutically active agent is in at least two dosage forms. In one embodiment, the at least two dosage forms are co-packaged together into a single kit.

In one embodiment of the pharmaceutical combination of the present disclosure, at least one of the dosage forms comprises a solid dispersion formulation. In one embodiment, the solid dispersion formulation is formed by solvent evaporation, hot-melt extrusion or spray drying dispersion.

In one embodiment of the pharmaceutical combination of the present disclosure, the solid dispersion formulation comprises one or more polymers selected from the group consisting of polyethylene glycol (PEG), polyvinyl pyrrolidone (PVP), polyethyleneoxide (PEO), poly(vinyl pyrrolidone-co-vinyl acetate) (PVP-VA), polymethacrylate, polyoxyethylene alkyl ether, polyoxyethylene-polyoxypropylene block copolymer, polyoxyethylene castor oil, polycaprolactam, polylactic acid, polyglycolic acid, poly(lactic-glycolic)acid, lipid, cellulose, pullulan, dextran, dextran acetate, dextran propionate, dextran succinate, dextran acetate propionate, dextran acetate succinate, dextran propionate succinate, dextran acetate propionate succinate, maltodextrin, hyaluronic acid, polysialic acid, chondroitin sulfate, heparin, fucoidan, pentosan polysulfate, spirulan, hydroxymethyl ethylcellulose, hydroxypropyl methylcellulose (HPMC), methyl cellulose, ethyl cellulose, hydroxyethyl cellulose, hydroxypropyl cellulose, carboxymethyl cellulose, carboxymethyl ethylcellulose (CMEC), sodium carboxymethyl cellulose, cellulose acetate succinate (CAS), methyl cellulose acetate succinate (MCAS), hydroxypropyl methylcellulose acetate succinate (HPMCAS), hydroxypropyl methylcellulose propionate succinate, hydroxypropyl methylcellulose propionate phthalate, cellulose acetate phthalate (CAP), hydroxypropyl methyl cellulose phthalate (HPMCP), hydroxypropyl methylcellulose acetate phthalate (HPMCAP), cellulose acetate trimellitate (CAT), hydroxypropyl methylcellulose acetate trimellitate (HPMCAT), hydroxypropyl methylcellulose propionate trimellitate, methyl cellulose acetate phthalate, hydroxypropyl cellulose acetate phthalate, cellulose acetate terephthalate, cellulose acetate isophthalate, cellulose acetate, cellulose butyrate, cellulose acetate butyrate, starch derivatives such as cyclodextrins (CDs), dextran polymer derivative, poly(methacrylic acid-co-methyl methacrylate) 1:1, poly(methacrylic acid-co-methyl methacrylate) 1:2, poly(methacrylic acid-co-ethyl acrylate) 1:1, and a graft copolymers comprised of polyethylene glycol, polyvinyl caprolactam and polyvinyl acetate, or any combinations thereof.

In one embodiment of the pharmaceutical combination of the present disclosure, Compound A and the second therapeutically active agent are in the same dosage form, and in the same solid dispersion formulation.

In one embodiment of the pharmaceutical combination of the present disclosure, Compound A and the second therapeutically active agent are in the same dosage form, but separate solid dispersion formulation. In one embodiment, each solid dispersion formulation each comprises different polymers.

In one embodiment of the pharmaceutical combination of the present disclosure, a daily dosage amount of Compound A is between about 50 mg and about 1500 mg, or between about 100 mg and about 1000 mg, or between about 200 mg and about 800 mg, or between about 300 mg and about 600 mg.

In one embodiment of the pharmaceutical combination of the present disclosure, an amount of Compound A per a dosage form is between about 5 mg and about 1000 mg, or between about 10 mg and about 500 mg, or between about 20 mg and about 250 mg, or between about 30 mg and about 300 mg, or between about 50 mg and about 200 mg.

In one embodiment of the pharmaceutical combination of the present disclosure, the second therapeutically active agent is enzalutamide, apalutamide, darolutamide, abiraterone, abiraterone acetate, methylprednisolone, or prednisone. In one embodiment, the second therapeutically active agent is enzalutamide.

In one embodiment of the pharmaceutical combination of Compound A and enzalutamide of the present disclosure, a daily dosage amount of enzalutamide is about 25 mg to about 550 mg, or about 50 mg to about 480 mg, or about 100 mg to about 400 mg, or about 200 mg to about 300 mg. In one embodiment, an amount of enzalutamide per a dosage form is about 5 mg to about 300 mg, or about 10 mg to about 200 mg, or about 30 mg to about 450 mg, or about 200 mg to about 300 mg.

In one embodiment of the pharmaceutical combination of Compound A and enzalutamide of the present disclosure, Compound A and enzalutamide are in the same composition. In one embodiment, the at least one composition comprises a solid dispersion formulation. In one embodiment, the at least one pharmaceutical composition in the combination comprises hydroxypropyl methylcellulose acetate succinate. In one embodiment, Compound A and enzalutamide are in a spray drying dispersion composition. In one embodiment, Compound A and enzalutamide are in the same composition and each are in a separate solid dispersion formulation comprising different polymers.

In one embodiment of the pharmaceutical combination of Compound A and enzalutamide of the present disclosure, enzalutamide is amorphous or crystalline or combinations thereof.

In one embodiment of the pharmaceutical combination of Compound A and enzalutamide of the present disclosure, Compound A and enzalutamide are in different compositions as a kit. In one embodiment, the kit comprises 1 to 6 compositions for each therapeutically active agent to be administered per day.

In one embodiment of the pharmaceutical combination of Compound A and enzalutamide of the present disclosure, at least one composition is a tablet. In one embodiment, Compound A and enzalutamide are in different layers or compartments of the same tablet composition. In one embodiment, the enzalutamide layer or the enzalutamide compartment of the composition is about 10% to about 70% by weight of the composition or about 20% to about 50% by weight of the composition, or about 30% to about 40% by weight of the composition; and the Compound A layer or the Compound A compartment is about 10% to about 70% by weight of the composition, or about 20% to about 50% by weight of the composition, or about 30% to about 40% by weight of the composition.

In one embodiment of the pharmaceutical combination of Compound A and enzalutamide of the present disclosure, the combination comprises a third therapeutically active agent. In one embodiment, the third therapeutically active agent is a CYP3A4 inhibitor. In one embodiment, the third therapeutically active agent is abiraterone or a pharmaceutically acceptable salt, solvate, or prodrug thereof.

In one embodiment of the pharmaceutical combination of the present disclosure, the second therapeutically active agent is darolutamide. In one embodiment, a daily dosage amount of darolutamide is about 200 mg to about 3000 mg, or about 300 mg to about 2000 mg, or about 400 mg to about 1500 mg, or about 700 mg to about 1200 mg. In one embodiment, an amount of darolutamide per a dosage form is about 20 mg to about 2000 mg, or about 50 mg to about 1000 mg, or about 100 mg to about 500 mg, or about 200 mg to about 300 mg.

In one embodiment of the pharmaceutical combination of Compound A and darolutamide of the present disclosure, Compound A and darolutamide and are in the same composition. In one embodiment, the at least one pharmaceutical composition comprises a solid dispersion formulation. In one embodiment, the at least one pharmaceutical composition in the combination comprises at least one polymer selected from polyvinyl pyrrolidone, polyethyleneoxide, or poly(vinyl pyrrolidone-co-vinyl acetate). In one embodiment, Compound A and darolutamide are in the same composition and each are in a separate solid dispersion formulation comprising different polymers. In one embodiment, Compound A and darolutamide are in the same composition and each are formulated separately.

In one embodiment of the pharmaceutical combination of Compound A and darolutamide of the present disclosure, darolutamide is amorphous or crystalline.

In one embodiment of the pharmaceutical combination of Compound A and darolutamide of the present disclosure, Compound A and darolutamide are in different compositions as a kit. In one embodiment, the kit comprises 1 to 6 compositions for each therapeutically active agent to be administered per day.

In one embodiment of the pharmaceutical combination of Compound A and darolutamide of the present disclosure, at least one composition is a tablet. In one embodiment, Compound A and enzalutamide and are in different layers or compartments of the same tablet composition. In one embodiment, the darolutamide layer or the darolutamide compartment in the tablet is about 10% to about 70% by weight of the composition, or about 20% to about 50% by weight of the composition, or about 30% to about 40% by weight of the composition; and the Compound A layer or the Compound A compartment is about 10% to about 70% by weight of the composition, or about 20% to about 50% by weight of the composition, or about 30% to about 40% by weight of the composition.

In one embodiment of the pharmaceutical combination of Compound A and darolutamide of the present disclosure, the combination comprises a third therapeutically active agent. In one embodiment, the third therapeutically active agent is a CYP3A4 inhibitor. In one embodiment, the third therapeutically active agent is abiraterone or a pharmaceutically acceptable salt, solvate, or prodrug thereof.

In one embodiment of the pharmaceutical combination of the present disclosure, the second therapeutically active agent is apalutamide. In one embodiment, a daily dosage amount of apalutamide is about 50 mg to about 500 mg, or about 100 mg to about 400 mg, or about 150 mg to about 300 mg, or about 175 mg to about 250 mg. In one embodiment, an amount of apalutamide per a dosage form is about 5 mg to about 100 mg, or about 10 mg to about 75 mg, or about 20 mg to about 60 mg, or about 25 mg to about 50 mg.

In one embodiment of the pharmaceutical combination of Compound A and apalutamide of the present disclosure, Compound A and apalutamide are in the same composition. In one embodiment, the at least one pharmaceutical composition comprises a solid dispersion formulation. In one embodiment, the at least one pharmaceutical composition in the combination comprises hydroxypropyl methylcellulose acetate succinate. In one embodiment, Compound A and apalutamide re in the same composition and each are in a separate solid dispersion formulation comprising different polymers.

In one embodiment of the pharmaceutical combination of Compound A and apalutamide of the present disclosure, apalutamide is amorphous or crystalline.

In one embodiment of the pharmaceutical combination of Compound A and apalutamide of the present disclosure, Compound A and apalutamide are in different compositions as a kit. In one embodiment, the kit comprises 1 to 6 compositions for each therapeutically active agent to be administered per day. In one embodiment, the composition is a tablet. In one embodiment, Compound A and apalutamide are in different layers or compartments of the tablet. In one embodiment, the apalutamide layer or the apalutamide compartment is about 10% to about 70% by weight of the composition, or about 20% to about 50% by weight of the composition, or about 30% to about 40% by weight of the composition; and the Compound A layer or the Compound A compartment is about 10% to about 70% by weight of the composition, or about 20% to about 50% by weight of the composition, or about 30% to about 40% by weight of the composition.

In one embodiment of the pharmaceutical combination of Compound A and apalutamide of the present disclosure, the combination comprises a third therapeutically active agent. In one embodiment, the third therapeutically active agent is a CYP3A4 inhibitor. In one embodiment, the third therapeutically active agent is abiraterone or a pharmaceutically acceptable salt, solvate, or prodrug thereof.

In one embodiment of the pharmaceutical combination of the present disclosure, the second therapeutically active agent is abiraterone or abiraterone acetate.

In one embodiment of the pharmaceutical combination of the present disclosure, the combination comprises abiraterone or abiraterone acetate as a third therapeutically active agent.

In one embodiment of the pharmaceutical combination of the present disclosure comprising abiraterone or abiraterone acetate, a daily dosage amount of abiraterone or abiraterone acetate is about 100 mg to about 2,000 mg, or about 200 mg to about 1,000 mg, or about 300 mg to about 800 mg, or about 400 mg to about 600 mg. In one embodiment, an amount of abiraterone or abiraterone acetate per a dosage form is about 50 mg to about 1,000 mg, or about 75 mg to about 800 mg, or about 100 mg to about 600 mg, or about 125 mg to about 500 mg.

In one embodiment of the pharmaceutical combination of the present disclosure comprising abiraterone or abiraterone acetate, Compound A and abiraterone or abiraterone acetate are in the same composition.

In one embodiment of the pharmaceutical combination of the present disclosure comprising abiraterone or abiraterone acetate, the at least one composition comprises a solid dispersion formulation.

In one embodiment of the pharmaceutical combination of the present disclosure comprising abiraterone or abiraterone acetate, Compound A and abiraterone or abiraterone acetate are in the same composition and each are in a separate solid dispersion formulation comprising different polymers.

In one embodiment of the pharmaceutical combination of the present disclosure comprising abiraterone or abiraterone acetate, abiraterone or abiraterone acetate is amorphous or crystalline.

In one embodiment of the pharmaceutical combination of the present disclosure comprising abiraterone or abiraterone acetate, Compound A and abiraterone or abiraterone acetate are in different compositions as a kit. In one embodiment, the kit comprises 1 to 6 compositions for each therapeutically active agent to be administered per day.

In one embodiment of the pharmaceutical combination of the present disclosure comprising abiraterone or abiraterone acetate, the at least one composition is a tablet. In one embodiment, Compound A and abiraterone or abiraterone acetate are in different layers or compartments of the tablet. In one embodiment, the abiraterone or abiraterone acetate layer or the abiraterone or abiraterone acetate compartment is about 10% to about 70% by weight of the composition, or about 20% to about 50% by weight of the composition, or about 30% to about 40% by weight of the composition; and the Compound A layer or the Compound A compartment is about 10% to about 70% by weight of the composition, or about 20% to about 50% by weight of the composition, or about 30% to about 40% by weight of the composition.

In one embodiment of the pharmaceutical combination of Compound A and abiraterone or abiraterone acetate of the present disclosure, the combination comprises a third therapeutically active agent. In one embodiment, the third therapeutically active agent is a CYP3A4 inhibitor. In one embodiment, the third therapeutically active agent is a steroid.

In one embodiment of the pharmaceutical combination of the present disclosure, the combination comprises a third therapeutically active agent. In one embodiment of the pharmaceutical combination of the present disclosure, the combination comprises a fourth therapeutically active agent. In one embodiment, the third therapeutically active agent is a steroid. In one embodiment, the fourth therapeutically active agent is a steroid.

In one embodiment of the pharmaceutical combination of the present disclosure comprising a steroid, the steroid is aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, mometasone, mometasone paramethasone, mometasone furoate monohydrate, prednicarbate, methylprednisolone, prednisolone, prednisone, tixocortol, tixocortol pivalate, triamcinolone, triamcinolone acetonide, or triamcinolone alcohol, or a pharmaceutically acceptable salt or solvate thereof. In one embodiment, the steroid is prednisone, prednisolone, or methylprednisolone.

In one embodiment of the pharmaceutical combination of the present disclosure comprising a steroid, a daily dosage amount of steroid is about 1 mg to about 50 mg, or about 2 mg to about 40 mg, or about 3 mg to about 30 mg, or about 5 mg to about 20 mg. In one embodiment, an amount of steroid per a dosage form is about 1 mg to about 30 mg, or about 2 mg to about 25 mg, or about 3 mg to about 15 mg, or about 4 mg to about 10 mg.

In one embodiment of the pharmaceutical combination of the present disclosure comprising a steroid, Compound A and the steroid are in the same or different composition.

In one embodiment of the pharmaceutical combination of the present disclosure comprising a steroid, the steroid is amorphous or crystalline.

In one embodiment of the pharmaceutical combination of the present disclosure comprising a steroid, Compound A and the steroid are in different compositions as a kit.

In one embodiment of the pharmaceutical combination of the present disclosure, the pharmaceutical combination comprises a kit comprising, one, two or three different dosage forms co-packaged together. In one embodiment, the kit which comprises 1 to 6 pharmaceutical compositions of each dosage form.

In one embodiment of the pharmaceutical combination of the present disclosure, all pharmaceutical compositions are co-packaged for daily administration.

In one embodiment of the pharmaceutical combination of the present disclosure, each pharmaceutical composition of each dosage form is for administration to a subject once every 24 hours, once every 12 hours, once every 8 hours, once every 6 hours, once every 5 hours, or once every 4 hours.

The present disclosure also relates to a pharmaceutical combination comprising darolutamide and a CYP3A4 inhibitor. In one embodiment, the CYP3A4 inhibitor is clarithromycin, telithromycin, erythromycin, nefazodone, atazanavir, darunavir, indinavir, lopinavir, nelfinavir, saquinavir, tipranavir, ritonavir, ketoconazole, itraconazole, fluconazole, verapamil, or cobicistat, or a pharmaceutically acceptable salt or solvate thereof.

The present disclosure also relates to a method for modulating androgen receptor activity, comprising administering any one of the pharmaceutical combinations and/or compositions as disclosed herein, to a subject in need thereof. In one embodiment, the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

The present disclosure also relates to a method for treating cancer, comprising administering any one of the pharmaceutical combinations and/or compositions as disclosed herein, to a subject in need thereof. In one embodiment, cancer is selected from prostate cancer, breast cancer, ovarian cancer, bladder cancer, pancreatic cancer, hepatocellular cancer, endometrial cancer, or salivary gland carcinoma.

In one embodiment of the methods of the present disclosure, the method is for treating prostate cancer. In one embodiment, prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer. In one embodiment, the prostate cancer is metastatic castration-resistant prostate cancer. In one embodiment, the prostate cancer expresses full-length androgen receptor or truncated androgen receptor splice variant. In one embodiment, the prostate cancer is resistant to enzalutamide monotherapy.

In one embodiment of the methods of the present disclosure, the method is for treating breast cancer. In one embodiment, the breast cancer is triple negative breast cancer.

### BRIEF DESCRIPTION OF THE DRAWINGS

FIG. 1A, FIG. 1B, and FIG. 1C show change in tumor volume in male SCID Beige mice bearing VCaP tumors after oral administration of enzalutamide, Compound A, or combination of enzalutamide and Compound A.

FIG. 2A and FIG. 2B shows individual tumor volume change from baseline measured at the end of experiment for oral administration of enzalutamide, Compound A, or combination of enzalutamide and Compound A to male SCID Beige mice bearing VCaP tumors.

FIG. 3 shows serum prostate-specific antigen (PSA) at the end of treatment with enzalutamide, Compound A, or combination of enzalutamide and Compound A in male SCID Beige mice bearing VCaP tumors.

FIG. 4 shows change in % body weight in male SCID Beige mice bearing VCaP tumors after oral administration of representative compounds.

FIG. 5A shows log-ratio of a gene's or a transcript's expression values in LNCaP cells treated with enzalutamide compared to synthetic androgen (R1881). FIG. 5B shows log-ratio of a gene's or a transcript's expression values in LNCaP cells treated with Compound A compared to synthetic androgen (R1881).

FIG. 6A shows relative mRNA expression of androgen responsive genes in LNCaP cells treated with enzalutamide (Enza), Compound A, or combinations of the two.

FIG. 6B shows the number of genes significantly down-regulated with Fold change of 4 or greater in LNCaP cells treated with enzalutamide (Enza), Compound A, or combinations of the two.

FIG. 6C shows the top 10 down-regulated genes in enzalutamide (Enza)/Compound A combination.

FIG. 7A shows the number of genes significantly down-regulated with Fold change of 4 or greater in LNCaP cells treated with apalutamide (Apa), darolutamide (Daro), Compound A, or combinations thereof.

FIG. 7B shows the top 10 down-regulated genes in darolutamide (Daro)/Compound A combination.

FIG. 8A shows log-ratio of a gene's or a transcript's expression values in LNCaP95 cells treated with enzalutamide compared to synthetic androgen (R1881). FIG. 8B shows log-ratio of a gene's or a transcript's expression values in LNCaP cells treated with Compound A compared to synthetic androgen (R1881).

FIG. 9A shows relative mRNA expression of androgen responsive genes in LNCaP95 (+R1881) cells treated with enzalutamide (Enza), Compound A, or combinations of the two.

FIG. 9B shows relative mRNA expression of androgen responsive genes in LNCaP95 (+R1881) cells treated with enzalutamide (Enza), Compound A, or combinations of the two.

FIG. 10 shows a heatmap from RNAseq analysis showing normalized expression of top 50 up- or down-regulated genes in response to R1881 stimulation when VCaP cells were treated with Compound A in combination with enzalutamide, darolutamide, or apalutamide.

FIG. 11A and FIG. 11B show normalized mRNA expression of androgen responsive genes in VCaP cells treated with Compound A in combination with enzalutamide, darolutamide, or apalutamide.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a androgen receptor modulator” refers to one or more androgen receptor modulators or at least one androgen receptor modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo. In one embodiment of the present disclosure, a prodrug includes a compound of for example abiraterone having a free hydroxyl group (—OH) that is acetylated (—OCOMe) or acylated at one or more positions.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The term “combination therapy” refers to a first therapy that includes Compound A in conjunction with a second therapy (e.g., therapy, surgery and/or an additional pharmaceutical agent) useful for treating, stabilizing, preventing, and/or delaying the disease or condition.

Administration in “conjunction with” another therapeutically active agent includes administration in the same or different composition(s) and/or combinations, either sequentially, simultaneously, or continuously, through the same or different routes. In some embodiments, the combination therapy optionally includes one or more pharmaceutically acceptable carriers or excipients, non-pharmaceutically active compounds, and/or inert substances.

The terms “pharmaceutical combination,” “therapeutic combination” or “combination” as used herein, refers to a single dosage form comprising at least two therapeutically active agents, or separate dosage forms comprising at least two therapeutically active agents together or separately for use in a combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration, including as a kit, or from two kits.

A “fixed dosage form” as used herein means a dosage formulation in which one or more therapeutically active agents are combined in a single dosage formulation.

A “co-packaged form” as used herein means that the therapeutically active agents are taken together, more than one dosage forms wherein the therapeutically active agents are taken together, or more than one dosage forms wherein the therapeutically active agents are taken separately in two or more pharmaceutical compositions, i.e., such as two or more separate tablets, capsules, gel capsules, pellets, etc, but typically the separate compositions are as a single kit.

As used herein, the term “pharmaceutical composition” refers to a formulation comprising at least one therapeutically active agent and a pharmaceutically acceptable excipient or carrier. A non-limiting example of pharmaceutical compositions includes tablets, capsules, gel capsules, syrup, liquid, gel, suspension, solid dispersion, or combinations thereof.

As used herein, the term “dosage form” refers to one or more pharmaceutical compositions which provides a specific amount of a therapeutically active agent, such as a unit dose. In one embodiment, a dosage form can be provided in one or more pharmaceutical compositions. For example, if a subject is to be administered with 200 mg of a therapeutically active agent at a time (unit dose), a dosage form can comprise two tablets each containing 100 mg of the therapeutically active agent, wherein the two tablets are the same pharmaceutical composition.

As used herein, the term “solid dispersion” is a system in a solid state (as opposed to a liquid or gaseous state) comprising at least two components, wherein one component is dispersed more or less evenly throughout the other component or components (homogenous mix). Generally, a solid dispersion formulation of a therapeutically active agent(s) refers to a dispersion mixture of the therapeutically active agent(s) in an inert carrier. Inert carriers can be a crystalline carrier (such as sugars), a polymeric carrier (such as HPMCAS), or a mixture of surfactants and polymers. Typically, a solid dispersion of a therapeutically active agent increases the surface area of the therapeutically active agent and enhances drug solubility and/or dissolution rate.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof

The following description includes information that may be useful in understanding the present invention. It is not an admission that any of the information provided herein is prior art or relevant to the presently claimed inventions, or that any publication specifically or implicitly referenced is prior art.

### Pharmaceutical Combination of the Disclosure

The present disclosure relates to pharmaceutical combinations and/or compositions comprising Compound A and one or more additional therapeutically active agents. In one embodiment, the pharmaceutical combinations and/or composition of the present disclosure is useful for treating various diseases and conditions including, but not limited to, cancer. In one embodiment, the pharmaceutical combinations and/or composition of the present disclosure is useful for treating prostate cancer.

The present disclosure also relates to pharmaceutical combinations and/or compositions comprising darolutamide and one or more additional therapeutically active agents. In one embodiment, the pharmaceutical combinations and/or compositions relate to darolutamide and a CYP3A4 inhibitor.

### Compound A

The present disclosure relates to pharmaceutical compositions or combinations comprising N-(4-((4-(2-(3-chloro-4-(2-chloroethoxy)-5-cyanophenyl)propan-2-yl)phenoxy) methyl)pyrimidin-2-yl)methanesulfonamideN-(4-((4-(2-(3-chloro-4-(2-chloroethoxy)-5-cyanophenyl) propan-2-yl) phenoxy)methyl)pyrimidin-2-yl)methanesulfonamide (Compound A), or a pharmaceutically acceptable salt, solvate, stereoisomer, or prodrug thereof. Compound A has the following structure:

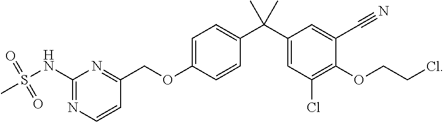 [SMILES: CC(C)(c1ccc(OCc2ccnc(NS(C)(=O)=O)n2)cc1)c1cc(Cl)c(OCCCl)c(C#N)c1]

Compound A is an androgen receptor modulator. Compound A binds to androgen receptor. Specifically, Compound A is an androgen receptor N-terminal domain inhibitor. Related androgen receptor modulator are disclosed in WO 2020/081999, which is incorporated by reference in its entirety for all purposes.

In one embodiment of the pharmaceutical combinations and/or compositions comprising Compound A, the one of more additional therapeutically active agent is an active agent useful for treating cancer, treating symptoms associated with cancer, or treating side effects caused by one or more therapeutically active agents. In one embodiment, the one of more additional therapeutically active agent is a metabolic inhibitors that may be beneficial to alter the dosing frequency or dosing amounts of Compound A and/or other therapeutically active agents present in the combination or the composition.

In one embodiment, the one or more additional therapeutically active agent is an androgen receptor ligand-binding domain inhibitor, a steroid, a CYP17 inhibitor, a CYP3A4 inhibitor, or an inhibitor of UGT enzymes.

### Androgen Receptor Ligand-binding Domain Inhibitors

In one embodiment, the additional therapeutically active agent is an androgen receptor ligand-binding domain inhibitor.

In one embodiment, the androgen receptor ligand-binding domain inhibitor is enzalutamide, apalutamide, darolutamide, bicalutamide, nilutamide, flutamide, ODM-204, or TAS3681, or a pharmaceutically acceptable salt or solvate thereof. In one embodiment, the androgen receptor ligand-binding domain inhibitor is enzalutamide, apalutamide, or darolutamide.

### Steroids

In one embodiment, the additional therapeutically active agent is a steroid.

In one embodiment, steroid is aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, mometasone, mometasone paramethasone, mometasone furoate monohydrate, prednicarbate, methylprednisolone, prednisolone, prednisone, tixocortol, tixocortol pivalate, triamcinolone, triamcinolone acetonide, or triamcinolone alcohol, or a pharmaceutically acceptable salt or solvate thereof. In one embodiment, steroid is prednisone, prednisolone, or methylprednisolone.

### CYP17 Inhibitors

In one embodiment, the additional therapeutically active agent is a CYP17 inhibitor.

In one embodiment, the CYP17 inhibitor is galeterone, abiraterone, or abiraterone acetate, or a pharmaceutically acceptable salt, solvate, or prodrug thereof. In one embodiment, abiraterone prodrug is abiraterone acetate. In one embodiment, the CYP17 inhibitor is abiraterone or abiraterone acetate.

### CYP3A4 Inhibitors

In one embodiment, the additional therapeutically active agent is a CYP3A4 inhibitor.

In one embodiment, the CYP3A4 inhibitor is clarithromycin, telithromycin, erythromycin, nefazodone, atazanavir, darunavir, indinavir, lopinavir, nelfinavir, saquinavir, tipranavir, ritonavir, ketoconazole, itraconazole, fluconazole, verapamil, or cobicistat, or a pharmaceutically acceptable salt or solvate thereof.

### Inhibitors of UGT Enzymes

In one embodiment, the additional therapeutically active agent is an uridine 5′-diphospho-glucuronosyltransferase (UDP-glucurono-syltransferase, UGT) inhibitor.

In one embodiment, the inhibitor of UGT enzyme is atazanavir, indinavir, lopinavir, nelfinavir, ritonavir, saquinavir, silybin, ritonavir, quinidine, diclofenac, everolimus, gemfibrozil, androsterone, phenylbutazone, ketoconazole, nilotinib, deoxyschizandrin, hecogenin, niflumic acid, efavirenz, or amitriptyline, or a pharmaceutically acceptable salt or solvate thereof.

### Representative Combination Embodiments

The present disclosure relates to pharmaceutical compositions and combinations comprising Compound A or a pharmaceutically acceptable salt, solvate, stereoisomer or prodrug thereof, and at least one additional therapeutically active agent. In one embodiment, the composition and/or combination comprises a second therapeutically active agent. In one embodiment, the composition and/or combination comprises a second therapeutically active agent and a third therapeutically active agent. In one embodiment, the composition and/or combination comprises a second therapeutically active agent, a third therapeutically active agent, and a fourth therapeutically active agent.

The present disclosure relates to a pharmaceutical combination comprising a therapeutically effective amount of a first therapeutically active agent Compound A or a pharmaceutically acceptable salt, solvate, stereoisomer or prodrug thereof, and a second therapeutically active agent in at least one pharmaceutical composition.

In one embodiment of the pharmaceutical combination of the present disclosure, the second therapeutically active agent is an androgen receptor ligand-binding domain inhibitor, a steroid, a CYP17 inhibitor, a CYP3A4 inhibitor, or an inhibitor of UGT enzyme as disclosed herein.

In one embodiment of the pharmaceutical combination of the present disclosure, the second therapeutically active agent is enzalutamide, apalutamide, darolutamide, abiraterone, abiraterone acetate, methylprednisolone, or prednisone. In one embodiment, the second therapeutically active agent is enzalutamide.

In one embodiment of the pharmaceutical combination of the present disclosure, the pharmaceutical combination comprises a third therapeutically active agent. In one embodiment, the third therapeutically active agent is a steroid as disclosed herein.

In one embodiment, the pharmaceutical combination relates to Compound A or a pharmaceutically acceptable salt, solvate, stereoisomer or prodrug thereof, and enzalutamide. In one embodiment, the pharmaceutical combination relates to Compound A or a pharmaceutically acceptable salt, solvate, stereoisomer or prodrug thereof, enzalutamide, and abiraterone or a pharmaceutically acceptable salt, solvate, or prodrug thereof. In one embodiment, the pharmaceutical combination relates to Compound A or a pharmaceutically acceptable salt, solvate, stereoisomer or prodrug thereof, enzalutamide, abiraterone or a pharmaceutically acceptable salt, solvate, or prodrug thereof, and a steroid. In one embodiment, the pharmaceutical combination relates to Compound A or a pharmaceutically acceptable salt, solvate, stereoisomer or prodrug thereof, enzalutamide, abiraterone or a pharmaceutically acceptable salt, solvate, or prodrug thereof, and a steroid selected from prednisone, prednisolone, or methylprednisolone. In one embodiment, the pharmaceutical combination further comprises one or more selected from an androgen receptor ligand-binding domain inhibitor, a steroid, a CYP17 inhibitor, a CYP3A4 inhibitor, or an inhibitor of UGT enzyme.

In one embodiment, the pharmaceutical combination relates to Compound A or a pharmaceutically acceptable salt, solvate, stereoisomer or prodrug thereof, and darolutamide. In one embodiment, the pharmaceutical combination relates to Compound A or a pharmaceutically acceptable salt, solvate, stereoisomer or prodrug thereof, darolutamide, and abiraterone or a pharmaceutically acceptable salt, solvate, or prodrug thereof. In one embodiment, the pharmaceutical combination relates to Compound A or a pharmaceutically acceptable salt, solvate, stereoisomer or prodrug thereof, darolutamide, abiraterone or a pharmaceutically acceptable salt, solvate, or prodrug thereof, and a steroid. In one embodiment, the pharmaceutical combination relates to Compound A or a pharmaceutically acceptable salt, solvate, stereoisomer or prodrug thereof, darolutamide, abiraterone or a pharmaceutically acceptable salt, solvate, or prodrug thereof, and a steroid selected from prednisone, prednisolone, or methylprednisolone. In one embodiment, the pharmaceutical combination further comprises one or more selected from an androgen receptor ligand-binding domain inhibitor, a steroid, a CYP17 inhibitor, a CYP3A4 inhibitor, or an inhibitor of UGT enzyme.

In one embodiment, the pharmaceutical combination relates to Compound A or a pharmaceutically acceptable salt, solvate, stereoisomer or prodrug thereof, and apalutamide. In one embodiment, the pharmaceutical combination relates to Compound A or a pharmaceutically acceptable salt, solvate, stereoisomer or prodrug thereof, apalutamide, and abiraterone or a pharmaceutically acceptable salt, solvate, or prodrug thereof. In one embodiment, the pharmaceutical combination relates to Compound A or a pharmaceutically acceptable salt, solvate, stereoisomer or prodrug thereof, apalutamide, abiraterone or a pharmaceutically acceptable salt, solvate, or prodrug thereof, and a steroid. In one embodiment, the pharmaceutical combination relates to Compound A or a pharmaceutically acceptable salt, solvate, stereoisomer or prodrug thereof, apalutamide, abiraterone or a pharmaceutically acceptable salt, solvate, or prodrug thereof, and a steroid selected from prednisone, prednisolone, or methylprednisolone. In one embodiment, the pharmaceutical combination further comprises one or more selected from an androgen receptor ligand-binding domain inhibitor, a steroid, a CYP17 inhibitor, a CYP3A4 inhibitor, or an inhibitor of UGT enzyme.

In one embodiment, the pharmaceutical combination relates to Compound A or a pharmaceutically acceptable salt, solvate, stereoisomer or prodrug thereof, and abiraterone or a pharmaceutically acceptable salt, solvate, or prodrug thereof. In one embodiment, the pharmaceutical combination relates to Compound A or a pharmaceutically acceptable salt, solvate, stereoisomer or prodrug thereof, abiraterone or a pharmaceutically acceptable salt, solvate, or prodrug thereof, and a steroid. In one embodiment, the pharmaceutical combination relates to Compound A or a pharmaceutically acceptable salt, solvate, stereoisomer or prodrug thereof, abiraterone or a pharmaceutically acceptable salt, solvate, or prodrug thereof, and a steroid selected from prednisone, prednisolone, or methylprednisolone. In one embodiment, the pharmaceutical combination further comprises one or more selected from an androgen receptor ligand-binding domain inhibitor, a steroid, a CYP17 inhibitor, a CYP3A4 inhibitor, or an inhibitor of UGT enzyme.

The present disclosure relates to a pharmaceutical combination comprising a therapeutically effective amount of darolutamide or a pharmaceutically acceptable salt or solvate thereof, as a first therapeutically active agent, and a CYP3A4 inhibitor as a second therapeutically active agent in at least one pharmaceutical composition. In one embodiment, the CYP3A4 inhibitor is arithromycin, telithromycin, erythromycin, nefazodone, atazanavir, darunavir, indinavir, lopinavir, nelfinavir, saquinavir, tipranavir, ritonavir, ketoconazole, itraconazole, fluconazole, verapamil, or cobicistat, or a pharmaceutically acceptable salt or solvate thereof.

### Therapeutic Use

The pharmaceutical compositions and combinations of the present disclosure find use in any number of methods. For example, in some embodiments the compounds are useful in methods for modulating androgen receptor (AR). In some embodiments, modulating androgen receptor (AR) activity is in a mammalian cell. In some embodiments, modulating androgen receptor (AR) can be in a subject in need thereof (e.g., a mammalian subject) and for treatment of any of the described conditions or diseases.

In one embodiment, the modulating AR is binding to AR. In other embodiments, the modulating AR is inhibiting AR.

In one embodiment, the modulating AR is modulating AR N-terminal domain (NTD). In one embodiment, the modulating AR is modulating AR NTD and AR ligand-binding domain (LBD). In one embodiment, the modulating AR is binding to AR NTD. In one embodiment, the modulating AR is binding to AR NTD and AR LBD. In other embodiments, the modulating AR is inhibiting AR NTD. In other embodiments, the modulating AR is inhibiting AR NTD and AR LBD. In some embodiments, modulating the AR is inhibiting transactivation of androgen receptor N-terminal domain (NTD).

In one embodiment of the present disclosure, methods for modulating androgen receptor activity, comprising administering any one of the pharmaceutical combination and/or composition as disclosed herein, to a subject in need thereof, are provided. In other embodiments, modulating androgen receptor (AR) activity is for treatment of at least one indication selected from the group consisting of: prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example in some embodiments, the indication is prostate cancer. In other embodiments, the prostate cancer is primary/localized prostate cancer, locally advanced prostate cancer, recurrent prostate cancer, metastatic prostate cancer, advanced prostate cancer, or metastatic castration-resistant prostate cancer (CRPC), or hormone-sensitive prostate cancer. While in other embodiments, the prostate cancer is androgen dependent prostate cancer. In other embodiments, the spinal and bulbar muscular atrophy is Kennedy's disease.

In one embodiment of the present disclosure, a method of treating a condition associated with cell proliferation in a patient in need thereof is provided. In one embodiment, the present invention provides a method of treating cancer or tumors, comprising administering any one of the pharmaceutical combination and/or composition as disclosed herein, to a subject in need thereof. In one embodiment, cancer is selected from prostate cancer, breast cancer, ovarian cancer, bladder cancer, pancreatic cancer, hepatocellular cancer, endometrial cancer, or salivary gland carcinoma.

In one embodiment of the methods of the present disclosure, the method is for treating prostate cancer. In one embodiment, prostate cancer is primary or localized prostate cancer, locally advanced prostate cancer, recurrent prostate cancer, advanced prostate cancer, metastatic prostate cancer, metastatic castration-resistant prostate cancer, and hormone-sensitive prostate cancer. In one embodiment, the prostate cancer is metastatic castration-resistant prostate cancer. In one embodiment, the prostate cancer expresses full-length androgen receptor or truncated androgen receptor splice variant. In one embodiment, the prostate cancer is resistant to enzalutamide monotherapy.

In one embodiment of the methods of the present disclosure, the method is for treating breast cancer. In one embodiment, the breast cancer is triple negative breast cancer.

In one embodiment of the present disclosure, a method of reducing, inhibiting, or ameliorating cell proliferation in a patient in need thereof is provided. In one embodiment, the reducing, inhibiting, or ameliorating in the method disclosed herein, is in vivo. In another embodiment, the reducing, inhibiting, or ameliorating is in vitro.

In one embodiment, the cells in the method disclosed herein, are a cancer cells. In one embodiment, the cancer cells are a prostate cancer cells. In one embodiment, the prostate cancer cells are cells of primary/localized prostate cancer (newly diagnosed or early stage), locally advanced prostate cancer, recurrent prostate cancer (e.g., prostate cancer which was not cured with primary therapy), metastatic prostate cancer, advanced prostate cancer (e.g., after castration for recurrent prostate cancer), metastatic castration-resistant prostate cancer (CRPC), or hormone-sensitive prostate cancer. In another embodiment, the prostate cancer cells are cells of a metastatic castration-resistant prostate cancer. In other embodiments, the prostate cancer cells are an androgen-dependent prostate cancer cells or an androgen-independent prostate cancer cells. In one embodiment, the cancer cells are breast cancer cells.

In one embodiment, the condition or disease associated with cell proliferation is cancer. In one embodiment of any one of the methods disclosed herein, the cancer is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In one embodiment, the condition or disease is prostate cancer. In one embodiment, prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In another embodiment, the prostate cancer is a metastatic castration-resistant prostate cancer. In some embodiments, the prostate cancer is an androgen-dependent prostate cancer cells or an androgen-independent prostate cancer. In one embodiment, the condition or disease is breast cancer. In one embodiment, the breast cancer is AR-positive triple negative breast cancer.

In another embodiment of the present disclosure, a method for reducing or preventing tumor growth, comprising contacting tumor cells with a pharmaceutical composition or a combination as disclosed herein.

In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth. In one embodiment, reducing or preventing tumor growth includes reduction in the rate of tumor growth such that the rate of tumor growth before treating a patient with the methods disclosed herein (r1) is faster than the rate of tumor growth after said treatment (r2) such that r1>r2.

In one embodiment, the reducing or preventing in the methods disclosed herein is in vivo.

In another embodiment, the treating is in vitro.

In one embodiment, the tumor cell in the method disclosed herein is selected from prostate cancer, breast cancer, ovarian cancer, endometrial cancer, or salivary gland carcinoma. In one embodiment, the tumor cells are prostate cancer tumor cells. In one embodiment, the prostate cancer tumor cells are tumor cells of primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the tumor cells are is breast cancer tumor cells.

### Therapeutic Use Related to Androgen Receptor Driven Gene Expression

In one embodiment, the present disclosure provides a method for treating a subject having a cancer, comprising, obtaining a sample of the cancer before and/or after treatment of the subject with an androgen receptor modulator.

In one embodiment of the present disclosure, a method of treating a patient with abnormal androgen receptor driven gene activity with androgen receptor modulator alone or in combination with a second therapeutic agent is provided.

In one embodiment, the present disclosure provides a method for treating a subject having a cancer, comprising, obtaining a sample of the cancer before treatment with an androgen receptor modulator, and determining in the sample, the expression level of an androgen receptor driven genes. In another specific embodiment, after testing the expression level of androgen receptor driven genes, the subject is administered an androgen receptor modulator alone and or in combination with a second therapeutically active agent as disclosed herein. In a specific embodiment, the genes are one or more selected from the group consisting of KLK2, FKBP5, TMPRSS2, KLK3, NCAPD3, NKX3-1, NDRG1, STEAP4, FAMOSA, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR 15L.

In one embodiment, the present disclosure provides a method of treating cancer in a subject having abnormal gene expression of one or more androgen receptor driven genes, comprising administering to the subject Compound A or a pharmaceutically acceptable salt, solvate, stereoisomer or prodrug thereof in combination with at least one additional therapeutically active agent. In one embodiment of any one of the methods disclosed herein, the androgen receptor driven gene is an androgen receptor full-length driven gene. In one embodiment, the androgen receptor driven gene is an androgen receptor V7 driven gene. In one embodiment of any one of the methods disclosed herein, the gene with an abnormal activity is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L. In one embodiment of the methods disclosed herein, cancer is selected from prostate cancer, breast cancer, ovarian cancer, endometrial cancer, or salivary gland carcinoma. In one embodiment, the cancer is prostate cancer. In one embodiment, the prostate cancer is selected from primary/localized prostate cancer, locally advanced prostate cancer, recurrent prostate cancer, metastatic prostate cancer, advanced prostate cancer, metastatic castration-resistant prostate cancer (CRPC), or hormone-sensitive prostate cancer. In other embodiments, the prostate cancer is a metastatic castration-resistant prostate cancer. In some embodiments, the prostate cancer is androgen-dependent prostate cancer or androgen-independent prostate cancer. In another embodiment, the cancer is breast cancer.

In one embodiment of any one of the methods as disclosed herein, the at least one additional therapeutically active agent is a nonsteroidal antiandrogen (NSAA). In one embodiment, the at least one additional therapeutically active agent is an AR LBD inhibitor. In one embodiment, the AR LBD inhibitor is enzalutamide, apalutamide, darolutamide, bicalutamide, nilutamide, flutamide, ODM-204, or TAS3681. In one embodiment, the AR LBD inhibitor is enzalutamide, apalutamide, or darolutamide.

In one embodiment of any one of the methods as disclosed herein, the at least one additional therapeutically active agent is an AR LBD inhibitor, a steroid, a CYP17 inhibitor, a CYP3A4 inhibitor, or an inhibitor of UGT enzymes.

In one embodiment, the present disclosure provides a method for treating a subject having a cancer, comprising, obtaining a sample of the cancer after treatment with an androgen receptor modulator, and determining, in the sample, the expression level of an androgen receptor driven gene, where if the gene expression level, when compared to a reference standard level, is decreased before or after treatment with the androgen receptor modulator, then proceeding with or resuming treatment of the subject with a therapeutically effective amount of Compound A and at least one additional therapeutically active agent. In a specific embodiment, the gene is selected from one or more of the group consisting of KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, and PRR 15L. In one embodiment, an androgen receptor modulator administered before the sample of cancer is obtained can be the same or different from Compound A.

In one embodiment, the at least one additional therapeutically active agent is an androgen receptor ligand-binding domain inhibitor selected from enzalutamide, apalutamide, darolutamide, bicalutamide, nilutamide, flutamide, ODM-204, or TAS3681. In one embodiment, the androgen receptor ligand-binding domain inhibitor is enzalutamide.

In one embodiment, the present disclosure provides a method for treating a subject having a cancer, comprising, obtaining a sample of the cancer after treatment with an androgen receptor modulator, and determining, in the sample, the expression level of an androgen receptor driven genes, where if the gene expression level, when compared to a reference standard level, is decreased before or after treatment with the androgen receptor modulator, then proceeding with or resuming treatment of the subject with a therapeutically effective amount of Compound A and enzalutamide, wherein the gene is selected from KLK2, FKBP5, TMPRSS2, KLK3, NCAPD3, NKX3-1, NDRG1, STEAP4, FAM105A, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L.

In one embodiment, the present disclosure provides a method for treating a subject having a cancer, comprising, obtaining a sample of the cancer after treatment with an androgen receptor modulator, and determining, in the sample, the expression level of an androgen receptor driven genes, where if the gene expression level, when compared to a reference standard level, is decreased before or after treatment with the androgen receptor modulator, then proceeding with or resuming treatment of the subject with a therapeutically effective amount of i) Compound A and enzalutamide, ii) Compound A and apalutamide, iii) Compound A and darolutamide, iv) Compound A and abiraterone or a pharmaceutically acceptable salt, solvate, or prodrug thereof, v) Compound A, enzalutamide, and abiraterone or a pharmaceutically acceptable salt, solvate, or prodrug thereof, vi) Compound A, apalutamide, and abiraterone or a pharmaceutically acceptable salt, solvate, or prodrug thereof, or vii) Compound A, darolutamide, and abiraterone or a pharmaceutically acceptable salt, solvate, or prodrug thereof, wherein the gene is selected from KLK2, FKBP5, TMPRSS2, KLK3, NCAPD3, NKX3-1, NDRG1, STEAP4, FAMOSA, AKAP12, PMEPA1, PLPP1, SNAl2, ACSL3, ERRFl1, CDC6, ELL2, CENPN, RHOU, EAF2, SGK1, SLC16A6, TIPARP, IGF1R, CCND1, ADAMTS1, or PRR15L. In one embodiment, the combination treatment further comprises one or more steroids. In one embodiment, the steroid is prednisone, prednisolone, or methylprednisolone. In one embodiment, the combination treatment further comprises one or more therapeutically active agents. In one embodiment, the one or more therapeutically active agent is an AR LBD inhibitor, a steroid, a CYP17 inhibitor, a CYP3A4 inhibitor, or an inhibitor of UGT enzymes.

### Pharmaceutical Compositions, Formulations, and Dosage Forms

The pharmaceutical combination of the present disclosure can be presented in various forms of pharmaceutical compositions. In one embodiment, the pharmaceutical combination comprising a therapeutically effective amount of a first therapeutically active agent Compound A or a pharmaceutically acceptable salt, solvate, stereoisomer or prodrug thereof, and a second therapeutically active agent is provided in at least one pharmaceutical composition.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., oncology and neurology disorders. In various aspects, the route of administration is systemic, e.g., oral or by injection. The agents or compounds, or pharmaceutically acceptable salts or derivatives thereof, are administered orally, nasally, transdermally, pulmonary, inhalationally, buccally, sublingually, intraperintoneally, subcutaneously, intramuscularly, intravenously, rectally, intrapleurally, intrathecally, intraportally, and parenterally. Alternatively or in addition, the route of administration is local, e.g., topical, intra-tumor and peri-tumor. In some embodiments, the compound is administered orally.

In certain embodiments, a pharmaceutical composition of the present disclosure is prepared for oral administration. In certain of such embodiments, a pharmaceutical composition is formulated by combining one or more agents and pharmaceutically acceptable carriers. Certain of such carriers enable pharmaceutical compositions to be formulated as tablets, pills, dragees, capsules, gel capsules, liquids, gels, syrups, slurries, suspensions and the like, for oral ingestion by a subject.

In one embodiment of the pharmaceutical combination of the present disclosure, the combination of Compound A and the second therapeutically active agent is in the same dosage form.

In one embodiment of the pharmaceutical combination of the present disclosure, the combination of Compound A and the second therapeutically active agent is in a single fixed dosage form. In one embodiment, a single tablet or a single capsule fixed dosage form comprises unit doses of both Compound A and the second therapeutically active agent. In one embodiment, a single dosage form comprises two or more tablets or two or more capsules, each comprising a fixed-dose of Compound A and the second therapeutically active agent.

In one embodiment, the combination of Compound A and the second therapeutically active agent is provided in at least two dosage forms or at least two pharmaceutical compositions. In one embodiment, the at least two dosage forms or the at least two pharmaceutical compositions are co-packaged together into a single kit. In one embodiment, Compound A is provided in one dosage form or one pharmaceutical composition and the second therapeutically active agent is provided in another dosage form or another pharmaceutical composition. In one embodiment, a single kit comprises Compound A in one dosage form or a pharmaceutical composition and the second therapeutically active agent in another dosage form or a pharmaceutical composition. In one embodiment, a single kit comprises Compound A formulated into one or more tablets or capsules and the second therapeutically active agent in different tablets or capsules. In one embodiment, a single kit comprises Compound A formulated into one or more tablets and the second therapeutically active agent in different tablets.

In one embodiment, a single kit comprises a single dose of Compound A and the second therapeutically active agent. In one embodiment, a single kit comprises a daily dose of Compound A and the second therapeutically active agent. In one embodiment, a daily dose may comprise one or more single doses of Compound A and/or the second therapeutically active agent to be taken at one, two, three, or four different times of the day. In one embodiment, Compound A and the second therapeutically active agent has the same dosing frequency (e.g., once a day, twice a day, once a week). In one embodiment, Compound A and the second therapeutically active agent has the same dosing frequency but taken at different times of the day. In one embodiment, Compound A and the second therapeutically active agent has the same dosing frequency and taken at the same time of the day. In one embodiment, Compound A and the second therapeutically active agent has a different dosing frequency (e.g., Compound A is taken once a day and the second therapeutically active agent is taken twice a day).

In one embodiment of the pharmaceutical combination of the present disclosure, the combination of Compound A, the second therapeutically active agent, and the third active agent is in a single fixed dosage form. In one embodiment, a single tablet or a single capsule fixed dosage form comprises unit doses of Compound A, the second therapeutically active agent, and the third therapeutically active agent. In one embodiment, a single dosage form comprises two or more tablets or capsules, each comprising a fixed-dose of Compound A, the second therapeutically active agent, and the third therapeutically active agent.

In one embodiment, the combination of Compound A, the second therapeutically active agent, and the third therapeutically active agent is provided in at least two dosage forms or at least two pharmaceutical compositions. In one embodiment, the combination is provided in at least three dosage forms. In one embodiment, the at least two dosage forms or the at least two pharmaceutical compositions are co-packaged together into a single kit. In one embodiment, Compound A is provided in one dosage form or one pharmaceutical composition, the second therapeutically active agent is provided in another dosage form or another pharmaceutical composition, and the third therapeutically active agent is provided in yet another dosage form or pharmaceutical composition. In one embodiment, Compound A and the second therapeutically active agent are provided in one dosage form or pharmaceutical composition, and the third therapeutically active agent is provided in another dosage form or pharmaceutical composition. In one embodiment, Compound A and the third therapeutically active agent are provided in one dosage form or pharmaceutical composition, and the second therapeutically active agent is provided in another dosage form or pharmaceutical composition. In one embodiment, the second and the third therapeutically active agents are provided in one dosage form or pharmaceutical composition, and Compound A is provided in another dosage form or pharmaceutical composition.

In one embodiment, a single kit comprises Compound A in one dosage form or a pharmaceutical composition, the second therapeutically active agent in another dosage form or a pharmaceutical composition, and the third therapeutically active agent in yet another dosage form or a pharmaceutical composition. In one embodiment, a single kit comprises Compound A formulated into one or more tablets or capsules, the second therapeutically active agent in different tablets or capsules, and the third therapeutically active agent in yet different tablets or capsules. In one embodiment, a single kit comprises Compound A and the second therapeutically active agent formulated into one or more tablets or capsules and the third therapeutically active agent in different tablets or capsules. In one embodiment, a single kit comprises Compound A and the third therapeutically active agent formulated into one or more tablets or capsules and the second therapeutically active agent in different tablets or capsules. In one embodiment, a single kit comprises the second and the third therapeutically active agent formulated into one or more tablets or capsules and Compound A in different tablets or capsules. In one embodiment, a single kit comprises Compound A formulated into one or more tablets, the second therapeutically active agent in different tablets, and the third therapeutically active agent in yet different tablets.

In one embodiment, when a fourth therapeutically active agent is present in the combination, the fourth therapeutically active agent is provided as separate dosage form or pharmaceutical composition and provided together with any one of the combinations disclosed above. In one embodiment, the fourth therapeutically active agent is formulated together with Compound A, the second and/or the third therapeutically active agent.

In one embodiment of the pharmaceutical combination of the present disclosure, the pharmaceutical combination comprises a kit comprising, one, two or three different dosage forms co-packaged together. Different dosage forms in a single co-package can comprise different therapeutically active agents. In some embodiments, all therapeutically active agents (Compound A, the second, optionally the third, optionally the fourth therapeutically active agents, and so forth) are provided in different dosage forms. In some embodiments, two or more therapeutically active agents are formulated into the same dosage form. In one embodiment, the kit can comprises 1, 2, 3, 4, 5, or 6 pharmaceutical compositions of each dosage form.

In one embodiment of the pharmaceutical combination of the present disclosure, the third therapeutically active agent is an androgen receptor ligand-binding domain inhibitor, a steroid, a CYP17 inhibitor, a CYP3A4 inhibitor, or an inhibitor of UGT enzyme. In one embodiment, the third therapeutically active agent is a steroid or a CYP3A4 inhibitor.

In one embodiment of the pharmaceutical combination of the present disclosure, the fourth therapeutically active agent is an androgen receptor ligand-binding domain inhibitor, a steroid, a CYP17 inhibitor, a CYP3A4 inhibitor, or an inhibitor of UGT enzyme. In one embodiment, the fourth therapeutically active agent is a steroid or a CYP3A4 inhibitor.

In one embodiment of the pharmaceutical combination of the present disclosure, all pharmaceutical compositions are co-packaged for daily administration.

In one embodiment of the pharmaceutical combination of the present disclosure, each pharmaceutical composition of each dosage form is for administration to a subject once every 24 hours, once every 12 hours, once every 8 hours, once every 6 hours, once every 5 hours, or once every 4 hours. In one embodiment, different therapeutically active agents in the combination can have different dosing schedule.

In one embodiment of the pharmaceutical combination of the present disclosure, at least one of the dosage forms or the pharmaceutical composition comprises a solid dispersion formulation. In one embodiment, the solid dispersion formulation is formed by solvent evaporation (also known as solvent processing), hot-melt extrusion or spray drying methods.

In one embodiment, a solid dispersion formulation is prepared by solvent evaporation where after the therapeutically active agent and the polymeric carrier are both dissolved in the same solvent or a solvent mixture, the solvent or the solvent mixture is rapidly removed by evaporation or by mixing with a non-solvent. Rapid removal of the solvent or the solvent mixture can be achieved using spray-drying, spray-coating (pan-coating, fluidized bed coating, etc.), and precipitation by rapid mixing of the polymer and drug solution with CO₂, water, or other non-solvent. In one embodiment, the solid dispersion is a supersaturated solid solution where the concentration of the therapeutically active agent in the polymeric carrier is above its equilibrium value.

In one embodiment of the pharmaceutical combination of the present disclosure, at least one of the dosage forms comprises a solid dispersion formulation, wherein the solid dispersion formulation has a single glass transition temperature. A single glass transition temperature for a solid dispersion indicates a high degree of homogeneity.

In one embodiment of the pharmaceutical combination of the present disclosure, the solid dispersion formulation comprises one or more water-soluble polymer. In one embodiment, the solid dispersion formulation comprises one or more cellulose derivatives. In one embodiment, the solid dispersion formulation comprises one or more water-soluble cellulosic polymer. In one embodiment, the solid dispersion formulation comprises one or more cellulosic or non-cellulosic polymer. In one embodiment, the solid dispersion formulation comprises one or more polymer that are neutral or ionizable in aqueous solution. In one embodiment, the solid dispersion formulation comprises one or more polymers that are ionizable and cellulosic. In one embodiment, the solid dispersion formulation comprises one or more polymers that are ionizable cellulosic polymers. In one embodiment, the solid dispersion formulation comprises one or more amphiphilic polymers. In one embodiment, the solid dispersion formulation comprises one or more hydrophilic polymer. In one embodiment, the solid dispersion formulation comprises one or more water-soluble hydrophilic polymer.

In one embodiment of the pharmaceutical combination of the present disclosure, the solid dispersion formulation comprises one or more polymers or polymeric carriers selected from polyethylene glycol (PEG), polyvinyl pyrrolidone (PVP), polyethyleneoxide (PEO), poly(vinyl pyrrolidone-co-vinyl acetate) (PVP-VA), polymethacrylate, polyoxyethylene alkyl ether, polyoxyethylene-polyoxypropylene block copolymer, polyoxyethylene castor oil, polycaprolactam, polylactic acid, polyglycolic acid, poly(lactic-glycolic)acid, lipid, cellulose, pullulan, dextran, dextran acetate, dextran propionate, dextran succinate, dextran acetate propionate, dextran acetate succinate, dextran propionate succinate, dextran acetate propionate succinate, maltodextrin, hyaluronic acid, polysialic acid, chondroitin sulfate, heparin, fucoidan, pentosan polysulfate, spirulan, hydroxymethyl ethylcellulose, hydroxypropyl methylcellulose (HPMC), methyl cellulose, ethyl cellulose, hydroxyethyl cellulose, hydroxypropyl cellulose, carboxymethyl cellulose, carboxymethyl ethylcellulose (CMEC), sodium carboxymethyl cellulose, cellulose acetate succinate (CAS), methyl cellulose acetate succinate (MCAS), hydroxypropyl methylcellulose acetate succinate (HPMCAS), hydroxypropyl methylcellulose propionate succinate, hydroxypropyl methylcellulose propionate phthalate, cellulose acetate phthalate (CAP), hydroxypropyl methyl cellulose phthalate (HPMCP), hydroxypropyl methylcellulose acetate phthalate (HPMCAP), cellulose acetate trimellitate (CAT), hydroxypropyl methylcellulose acetate trimellitate (HPMCAT), hydroxypropyl methylcellulose propionate trimellitate, methyl cellulose acetate phthalate, hydroxypropyl cellulose acetate phthalate, cellulose acetate terephthalate, cellulose acetate isophthalate, cellulose acetate, cellulose butyrate, cellulose acetate butyrate, starch derivatives such as cyclodextrins (CDs), dextran polymer derivative, poly(methacrylic acid-co-methyl methacrylate) 1:1, poly(methacrylic acid-co-methyl methacrylate) 1:2, poly(methacrylic acid-co-ethyl acrylate) 1:1, or a graft copolymers comprised of polyethylene glycol, polyvinyl caprolactam and polyvinyl acetate, or any combinations thereof. In one embodiment, one or more polymers or polymeric carriers is PEG, polyvinyl pyrrolidone, polyethyleneoxide, poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic)acid, lipids, cellulose, pullulan, dextran, maltodextrin, hyaluronic acid, polysialic acid, chondroitin sulfate, heparin, fucoidan, pentosan polysulfate, spirulan, hydroxypropyl methyl cellulose, hydroxypropyl cellulose, 10 carboxymethyl ethylcellulose, hydroxypropyl methylcellulose acetate succinate (including from L, M, H, LF and/or LG HPMCAS) cellulose acetate phthalate, cellulose acetate trimellitate, ethyl cellulose, cellulose acetate, cellulose butyrate, cellulose acetate butyrate, ethyl cellulose and starch derivatives such as cyclodextrins (CDs), or dextran polymer derivative. In one embodiment, one or more polymers or polymeric carriers is hydroxypropyl methylcellulose acetate succinate. In one embodiment, hydroxypropyl methylcellulose acetate succinate (HPMCAS) has a grade L, M, H, LF, MF, HF, LG, MG, and/or HG.

In one embodiment, neutral non-cellulosic polymers or polymeric carriers are selected from vinyl polymers and copolymers having substituents of hydroxyl, alkylacyloxy, and cyclicamido polyvinyl alcohols that have at least a portion of their repeat units in the unhydrolyzed (vinyl acetate) form; polyvinyl alcohol polyvinyl acetate copolymers; polyvinyl pyrrolidone; polyvinylpyrrolidone vinyl acetate; or polyethylene polyvinyl alcohol copolymers.

In one embodiment, ionizable non-cellulosic polymers or polymeric carriers are selected from carboxylic acid-functionalized vinyl polymers, such as the carboxylic acid functionalized polymethacrylates and carboxylic acid functionalized polyacrylates such as the Eudragit® polymers; amine-functionalized polyacrylates and polymethacrylates; proteins; or carboxylic acid functionalized starches such as starch glycolate.

In one embodiment, amphiphilic non-cellulosic polymer or polymeric carrier is acrylate and methacrylate copolymers. Commercial grades of such copolymers include the Eudragit® polymers, which are copolymers of methacrylates and acrylates; and graft copolymers of polyethyleneglycol, polyvinylcaprolactam, and polyvinylacetate, such as Soluplus®.

In one embodiment, neutral non-ionizable cellulosic polymers or polymeric carriers are selected from hydroxypropyl methyl cellulose acetate, hydroxypropyl methyl cellulose, hydroxypropyl cellulose, methyl cellulose, hydroxyethyl methyl cellulose, hydroxyethyl cellulose acetate, or hydroxyethyl ethyl cellulose.

In one embodiment, neutral amphiphilic cellulosic polymers or polymeric carriers are selected from hydroxypropyl methyl cellulose or hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

In one embodiment, cellulosic polymers or polymeric carriers are selected from hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, or ethyl picolinic acid cellulose acetate. In one embodiment, cellulosic polymers or polymeric carriers are selected from cellulose acetate phthalate (CAP), methyl cellulose acetate phthalate, ethyl cellulose acetate phthalate, hydroxypropyl cellulose acetate phthalate, hydroxylpropyl methyl cellulose phthalate (HPMCP), hydroxypropyl methyl cellulose acetate phthalate (HPMCAP), hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

In one embodiment, cellulosic ionaizable polymers or polymeric carriers are selected from hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, or hydroxyethyl cellulose acetate succinate.

In one embodiment of the pharmaceutical combination of the present disclosure, at least one of the therapeutically active agent is provided as a solid dispersion. In one embodiment, the composition comprising the solid dispersion of the therapeutically active agent is about 30% to about 90% by weight of the total composition (which can include other pharmaceutically acceptable excipients and/or coatings), including all values and subranges therebetween. In one embodiment, the composition comprising the solid dispersion of the therapeutically active agent is about 30%, about 35%, about 40%, about 45%, about 50%, about 55%, about 60%, about 65%, about 70%, about 75%, about 80%, about 85%, or about 90% by weight of the total composition, including all values therebetween.

In one embodiment of the pharmaceutical combination of the present disclosure, at least one of the therapeutically active agent is provided as a solid dispersion where the therapeutically active agent is about 25% to about 85%, or about 50% to about 70% of the solid dispersion by weight, including all values and subranges therebetween. In one embodiment, the therapeutically active agent is provided as a solid dispersion where the therapeutically active agent is about 25%, about 30%, about 35%, about 40%, about 45%, about 50%, about 55%, about 60%, about 65%, about 70%, about 75%, about 80%, or about 85% of the solid dispersion by weight, including all values therebetween.

In one embodiment of the pharmaceutical combination of the present disclosure, at least one of the therapeutically active agent is provided as a solid dispersion where the ratio of the therapeutically active agent to the at least one polymer or polymeric carrier is about 5:1 to about 1:40, about 4:1 to about 1:20, about 3:1 to about 1:10, or about 2:1 to about 1:5, including all values and subranges therebetween. In one embodiment, the ratio of the therapeutically active agent to the at least one polymer or polymeric carrier in the solid dispersion is about 5:1, about 4.5:1, about 4:1, about 3.5:1, about 3:1, about 2.5:1, about 2:1, or about 1.5:1.

In one embodiment, a solid dispersion is a spray-dried dispersion (SDD).

In one embodiment of the pharmaceutical combination of the present disclosure, Compound A and the second therapeutically active agent are in the same solid dispersion formulation. In one embodiment, Compound A and the second therapeutically active agent are dispersed in the same inert carrier. In one embodiment, Compound A and the second therapeutically active agent are dispersed in the same polymeric carrier.

In one embodiment of the pharmaceutical combination of the present disclosure, Compound A and the second therapeutically active agent are in separate solid dispersion formulations. In one embodiment, the solid dispersion formulation comprising Compound A and the solid dispersion formulation comprising the second active agent uses different inert carriers. In one embodiment, the solid dispersion formulation comprising Compound A and the solid dispersion formulation comprising the second active agent uses different polymeric carriers. In one embodiment, the solid dispersion formulation comprising Compound A and the solid dispersion formulation comprising the second active agent uses the same polymeric carrier but formulated separately. In one embodiment, the solid dispersion formulation comprising Compound A and the solid dispersion formulation comprising the second active agent are provided in the same dosage form.

In one embodiment of the pharmaceutical combination of the present disclosure, Compound A and the second therapeutically active agent are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical combination of the present disclosure, at least one composition is a tablet. In one embodiment, Compound A and the second therapeutically active agent are in different layers or compartments of the same tablet composition. In one embodiment, the layers or compartments comprising the different therapeutically active agent is adjacent to one another. In one embodiment, the layers or compartments comprising the different therapeutically active agent are separated by one or more coatings or compartments such that the two therapeutically active agents do not come in contact. The one or more coatings or compartments separating the different therapeutically active agents can be functional (e.g., modifies release) or inert (e.g., just providing physical separation). In one embodiment, the Compound A layer or the Compound A compartment is about 10% to about 70% by weight of the composition, or about 20% to about 50% by weight of the composition, or about 30% to about 40% by weight of the composition, or any values or subranges therebetween. In one embodiment, the Compound A layer or the Compound A compartment is about 10%, about 15%, about 20%, about 25%, about 30%, about 35%, about 40%, about 45%, about 50%, about 55%, about 60%, about 65%, or about 70% weight of the composition, or any values therebetween. In one embodiment, the layer or the compartment of the composition comprising the second therapeutically active agent is about 10% to about 70% by weight of the composition or about 20% to about 50% by weight of the composition, or about 30% to about 40% by weight of the composition, or any values or subranges therebetween. In one embodiment, the layer or the compartment of the composition comprising the second therapeutically active agent is about 10%, about 15%, about 20%, about 25%, about 30%, about 35%, about 40%, about 45%, about 50%, about 55%, about 60%, about 65%, or about 70% weight of the composition, or any values therebetween.

In one embodiment of the pharmaceutical combination of the present disclosure, a daily dosage amount of Compound A is between about 50 mg and about 1500 mg, or between about 100 mg and about 1000 mg, or between about 200 mg and about 800 mg, or between about 300 mg and about 600 mg, or any values or subranges therebetween. In one embodiment, the daily dosage amount of Compound A is about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, or about 1500 mg, or any values therebetween. In one embodiment, the daily dose of Compound A is administered once a day, or divided into twice-a-day or three times a day doses. In one embodiment, the daily dose of Compound A is provided in one tablet or one capsule, or the daily dose is divided into two, three, four, five, or six tablets or capsules.

In one embodiment of the pharmaceutical combination of the present disclosure, an amount of Compound A per a dosage form is between about 5 mg and about 1000 mg, or between about 10 mg and about 500 mg, or between about 20 mg and about 250 mg, or between about 30 mg and about 300 mg, or between about 50 mg and about 200 mg, or any values or subranges therebetween. In one embodiment, an amount of Compound A per a dosage form is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values therebetween.

In one embodiment of the pharmaceutical combination of the present disclosure, an amount of Compound A per one tablet or one capsule is between about 5 mg and about 1000 mg, or between about 10 mg and about 500 mg, or between about 20 mg and about 250 mg, or between about 30 mg and about 300 mg, or between about 50 mg and about 200 mg, or any values or subranges therebetween. In one embodiment, an amount of Compound A per one tablet or one capsule is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values therebetween.

In one embodiment of the pharmaceutical combination of the present disclosure, a daily dosage amount of the second therapeutically active agent is about 25 mg to about 550 mg, or about 50 mg to about 480 mg, or about 100 mg to about 400 mg, or about 200 mg to about 300 mg, or any values or subranges therebetween. In one embodiment, an amount of the second therapeutically active agent per a dosage form is about 5 mg to about 300 mg, or about 10 mg to about 200 mg, or about 30 mg to about 450 mg, or about 200 mg to about 300 mg, or any values or subranges therebetween. In one embodiment, the second therapeutically active agent is an AR LBD inhibitor. In one embodiment of the pharmaceutical combination of the present disclosure, the second therapeutically active agent is enzalutamide, apalutamide, darolutamide, abiraterone, abiraterone acetate, methylprednisolone, or prednisone. In one embodiment, the second therapeutically active agent is enzalutamide.

### Compound A+Enzalutamide

In one embodiment of the pharmaceutical combination of the present disclosure, the second therapeutically active agent is enzalutamide and a daily dosage amount of enzalutamide is about 25 mg to about 550 mg, or about 50 mg to about 480 mg, or about 100 mg to about 400 mg, or about 200 mg to about 300 mg, or any values or subranges therebetween. In one embodiment, the daily dosage amount of enzalutamide is about 20 mg, about 25 mg, about 30 mg, about 40 mg, about 50 mg, about 60 mg, about 70 mg, about 80 mg, about 90 mg, about 100 mg, about 150 mg, about 160 mg, about 200 mg, about 250 mg, about 300 mg, about 350 mg, about 400 mg, about 450 mg, about 480 mg, about 500 mg, about 550 mg, or about 600 mg, or any values therebetween. In one embodiment, the daily dose of enzalutamide is administered once a day, or divided into twice-a-day or three times a day doses. In one embodiment, the daily dose of enzalutamide is provided in one tablet or one capsule, or the daily dose is divided into two, three, four, five, or six tablets or capsules.

In one embodiment of the pharmaceutical combination of the present disclosure, the second therapeutically active agent is enzalutamide and an amount of enzalutamide per a dosage form is about 5 mg to about 300 mg, or about 10 mg to about 200 mg, or about 30 mg to about 450 mg, or about 200 mg to about 300 mg, or any values or subranges therebetween. In one embodiment, the amount of enzalutamide per a dosage form is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg, or any values therebetween.

In one embodiment of the pharmaceutical combination of the present disclosure, the second therapeutically active agent is enzalutamide and an amount of enzalutamide per one tablet or one capsule form is about 5 mg to about 300 mg, or about 10 mg to about 200 mg, or about 30 mg to about 450 mg, or about 200 mg to about 300 mg, or any values or subranges therebetween. In one embodiment, the amount of enzalutamide per one tablet or one capsule is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg, or any values therebetween. In one embodiment, the amount of enzalutamide per one tablet or one capsule is about 40 mg or about 80 mg.

In one embodiment of the pharmaceutical combination of Compound A and enzalutamide of the present disclosure, Compound A and enzalutamide are in the same composition. In one embodiment, Compound A and enzalutamide are in different compositions. In one embodiment, at least one composition comprises a solid dispersion formulation. In one embodiment, at least one composition in the combination comprises hydroxypropyl methylcellulose acetate succinate. In one embodiment, hydroxypropyl methylcellulose acetate succinate is HPMCAS-M.

In one embodiment, a solid dispersion formulation comprising enzalutamide comprises one or more polymers or polymeric carriers selected from hydroxypropylmethylcellulose acetate succinate (HPMCAS), hydroxypropylmethylcellulose (HIPMC), hydroxypropylmethylcellulosephthalate (HPMCP), polyvinylpyrrolidonevinylacetate (PVP-VA), copolymers of methacrylic acid and methylmethacrylate (approximate 1:1 ratio) such as EUDRAGIT L-100®, and graft copolymers of polyethyleneglycol, polyvinylcaprolactam, and polyvinylacetate such as SOLUPLUS®. In one embodiment, a solid dispersion formulation comprising enzalutamide comprises one or more polymers or polymeric carriers selected from polyvinyl pyrrolidone (PVP), polyethyleneoxide (PEO), 5 poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic)acid, lipids, cellulose, pullulan, dextran, maltodextrin, hyaluronic acid, polysialic acid, chondroitin sulfate, heparin, fucoidan, pentosan polysulfate, spirulan, hydroxypropyl methyl cellulose (HPMC), hydroxypropyl cellulose (HPC), carboxymethyl ethylcellulose (CMEC), hydroxypropyl methylcellulose acetate succinate (HPMCAS), cellulose acetate phthalate (CAP), cellulose acetate trimellitate (CAT), ethyl cellulose, cellulose acetate, cellulose butyrate, cellulose acetate butyrate, dextran polymer derivatives, and pharmaceutically acceptable forms, derivatives. In one embodiment, a solid dispersion formulation comprising enzalutamide comprises HPMCAS.

In one embodiment, a solid dispersion formulation comprising enzalutamide comprises one or more pharmaceutically acceptable excipient selected from sulfonated hydrocarbons and their salts, such as dioctylsodiumsulfocuccinate and sodium laurylsulfate; polyoxyethylene sorbitan fatty acid esters, such as polysorbate-80 and polysorbate-20; polyoxyethylene alkyl ethers; polyoxyethylene castor oil; polyoxyethylene (-40 or -60) hydrogenated castor oil; tocopheryl polyethyleneglycol 1000 succinate; glyceryl polyethyleneglycol-8 caprylate/caprate; polyoxyethylene-32 glyceryl laurate; polyoxyethylene fatty acid esters; polyoxyethylene-polyoxypropylene block copolymers; polyglycolized glycerides; long-chain fatty acids such as palmitic and stearic and oleic and ricinoleic acids; medium-chain and long-chain saturated and unsaturated mono-, di- and tri-glycerides and mixtures thereof; fractionated coconut oils; mono- and di-glycerides of capric and caprylic acids; bile salts such as sodium taurocholate; and phospholipids such as egg lecithin, soy lecithin, 1,2-diacyl-sn-glycerophosphorylcholines such as 1-palmitoyl-2-oleyl-sn-glycerophosphorylcholine, dipalmitoyl-sn-glycerophosphorylcholine, distearoyl-sn-glycerophosphorylcholine, or 1-palmitoyl-2-stearoyl-sn-glycerophosphorylcholine.

In one embodiment, the composition comprising enzalutamide comprises one or more pharmaceutically acceptable carrier or excipient selected from caprylocaproyl polyoxylglycerides, butylated hydroxyanisole, butylated hydroxytoluene, gelatin, sorbitol sorbitan solution, glycerin, purified water, titanium dioxide, or black iron oxide. In one embodiment, the composition comprising enzalutamide is a capsule. In one embodiment, the composition comprising enzalutamide is a soft gelatin capsule.

In one embodiment, the composition comprising enzalutamide comprises one or more pharmaceutically acceptable carrier or excipient selected from hydroxypropyl methylcellulose acetate succinate, microcrystalline cellulose, colloidal silicon dioxide, croscarmellose sodium, or magnesium stearate. In one embodiment, the composition comprising enzalutamide is a tablet. In one embodiment, the tablet comprising enzalutamide has a film-coat. In one embodiment, the film coat comprises one or more pharmaceutically acceptable excipient selected from hydroxypropyl methylcellulose, talc, polyethylene glycol, titanium dioxide, or ferric oxide. Examples of formulations of enzalutamide are disclosed in US2014/0179749 A1 and WO2015/118015 A1, the disclosures of which are hereby incorporated by reference in their entirety.

In one embodiment of the pharmaceutical combination of the present disclosure, Compound A and enzalutamide are in the same solid dispersion formulation. In one embodiment, Compound A and enzalutamide are dispersed in the same inert carrier. In one embodiment, Compound A and enzalutamide are dispersed in the same polymeric carrier.

In one embodiment of the pharmaceutical combination of the present disclosure, Compound A and enzalutamide are in separate solid dispersion formulations. In one embodiment, the solid dispersion formulation comprising Compound A and the solid dispersion formulation comprising enzalutamide uses different inert carriers. In one embodiment, the solid dispersion formulation comprising Compound A and the solid dispersion formulation comprising enzalutamide uses different polymeric carriers. In one embodiment, the solid dispersion formulation comprising Compound A and the solid dispersion formulation comprising enzalutamide uses the same polymeric carrier but formulated separately. In one embodiment, the solid dispersion formulation comprising Compound A and the solid dispersion formulation comprising enzalutamide are provided in the same dosage form.

In one embodiment of the pharmaceutical combination of the present disclosure, enzalutamide is amorphous or crystalline or combinations thereof. In one embodiment of the pharmaceutical combination of the present disclosure, enzalutamide is at least 60% amorphous. In one embodiment of the pharmaceutical combination of the present disclosure, the major portion of enzalutamide is amorphous which is less than 10% crystalline. In one embodiment of the pharmaceutical combination of the present disclosure, enzalutamide is substantially amorphous.

In one embodiment of the pharmaceutical combination of the present disclosure, Compound A and enzalutamide are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical combination of Compound A and enzalutamide of the present disclosure, at least one composition is a tablet. In one embodiment, Compound A and enzalutamide are in different layers or compartments of the same tablet composition. In one embodiment, the different layers or compartments comprising Compound A and enzalutamide are adjacent to one another. In one embodiment, the different layers or compartments comprising Compound A and enzalutamide are separated by one or more coatings or compartments such that the two therapeutically active agents do not come in contact. The one or more coatings or compartments separating the different therapeutically active agents can be functional (e.g., modifies release) or inert (e.g., just providing physical separation). In one embodiment, the Compound A layer or the Compound A compartment is about 10% to about 70% by weight of the composition, or about 20% to about 50% by weight of the composition, or about 30% to about 40% by weight of the composition, or any values or subranges therebetween. In one embodiment, the Compound A layer or the Compound A compartment is about 10%, about 15%, about 20%, about 25%, about 30%, about 35%, about 40%, about 45%, about 50%, about 55%, about 60%, about 65%, or about 70% weight of the composition, or any values therebetween. In one embodiment, the layer or the compartment of the composition comprising enzalutamide is about 10% to about 70% by weight of the composition or about 20% to about 50% by weight of the composition, or about 30% to about 40% by weight of the composition, or any values or subranges therebetween. In one embodiment, the layer or the compartment of the composition comprising enzalutamide is about 10%, about 15%, about 20%, about 25%, about 30%, about 35%, about 40%, about 45%, about 50%, about 55%, about 60%, about 65%, or about 70% by weight of the composition, or any values therebetween.

In one embodiment of the pharmaceutical combination of Compound A and enzalutamide of the present disclosure, the combination comprises a third therapeutically active agent. In one embodiment, the third therapeutically active agent is abiraterone or a pharmaceutically acceptable salt, solvate, or prodrug thereof.

In one embodiment of the pharmaceutical combination of Compound A and enzalutamide of the present disclosure, the combination comprises a third therapeutically active agent and a fourth therapeutically active agent. In one embodiment, the third therapeutically active agent is abiraterone or a pharmaceutically acceptable salt, solvate, or prodrug thereof, and the fourth therapeutically active agent is any one of the steroids as disclosed herein. In one embodiment, the fourth therapeutically active agent is prednisone, prednisolone, or methylprednisolone.

In one embodiment, the third therapeutically active agent is a CYP3A4 inhibitor.

### Compound A+Darolutamide

In one embodiment of the pharmaceutical combination of the present disclosure, the second therapeutically active agent is darolutamide and a daily dosage amount of darolutamide is about 200 mg to about 3000 mg, or about 300 mg to about 2000 mg, or about 400 mg to about 1500 mg, or about 700 mg to about 1200 mg, or any values or subranges therebetween. In one embodiment, the daily dosage amount of darolutamide is about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg, about 2,000 mg, about 2,050 mg, about 2,100 mg, about 2,150 mg, about 2,200 mg, about 2,250 mg, about 2,300 mg, about 2,350 mg, about 2,400 mg, about 2,450 mg, about 2,500 mg, about 2,550 mg, about 2,600 mg, about 2,650 mg, about 2,700 mg, about 2,750 mg, about 2,800 mg, about 2,850 mg, about 2,900 mg, about 2,950 mg, or about 3,000 mg, or any values therebetween. In one embodiment, the daily dose of darolutamide is administered once a day, or divided into twice-a-day or three times a day doses. In one embodiment, the daily dose of darolutamide is provided in one tablet or one capsule, or the daily dose is divided into two, three, four, five, or six tablets or capsules.

In one embodiment of the pharmaceutical combination of the present disclosure, the second therapeutically active agent is darolutamide and an amount of darolutamide per a dosage form is about 20 mg to about 2000 mg, or about 50 mg to about 1000 mg, or about 100 mg to about 500 mg, or about 200 mg to about 300 mg, or any values or subranges therebetween. In one embodiment, the amount of darolutamide per a dosage form is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 200 mg to about 3000 mg, or about 300 mg to about 2000 mg, or about 400 mg to about 1500 mg, or about 700 mg to about 1200 mg, or any values or subranges therebetween.

In one embodiment of the pharmaceutical combination of the present disclosure, the second therapeutically active agent is darolutamide and an amount of darolutamide per one tablet or one capsule form is about 20 mg to about 2000 mg, or about 50 mg to about 1000 mg, or about 100 mg to about 500 mg, or about 200 mg to about 300 mg, or any values or subranges therebetween.

In one embodiment, the amount of darolutamide per one tablet or one capsule is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 200 mg to about 3000 mg, or about 300 mg to about 2000 mg, or about 400 mg to about 1500 mg, or about 700 mg to about 1200 mg, or any values or subranges therebetween. In one embodiment, the amount of darolutamide per one tablet or one capsule is about 300 mg.

In one embodiment of the pharmaceutical combination of Compound A and darolutamide of the present disclosure, Compound A and darolutamide are in the same composition. In one embodiment, Compound A and darolutamide in the same composition is formulated together or formulated separately. In one embodiment, Compound A and darolutamide are in different compositions. In one embodiment, at least one composition comprises a solid dispersion formulation. In one embodiment, at least one composition in the combination comprises polyvinyl pyrrolidone, polyethyleneoxide, or poly(vinyl pyrrolidone-co-vinyl acetate). In one embodiment, at least one composition in the combination comprises polyvinyl pyrrolidone. In one embodiment, at least one composition in the combination comprises povidone K 30. In one embodiment, the composition comprising darolutamide comprises darolutamide in crystalline form, amorphous form, or a combination of the two forms. In one embodiment, the composition comprising darolutamide comprises darolutamide as intragranular crystalline form, extragranular crystalline form, or a combination of the two forms.

In one embodiment, the composition comprising darolutamide comprises one or more pharmaceutically acceptable carrier or excipient selected from calcium hydrogen phosphate, croscarmellose sodium, lactose monohydrate, magnesium stearate, povidone K 30, hypromellose 15 cP, macrogol 3350, or titanium dioxide. In one embodiment, the composition comprising darolutamide is a tablet. In one embodiment, the tablet comprising darolutamide has a film-coat. Examples of formulations of darolutamide are disclosed in WO2019/032840 A1, the disclosures of which are hereby incorporated by reference in their entirety.

In one embodiment of the pharmaceutical combination of the present disclosure, Compound A and darolutamide are in the same solid dispersion formulation. In one embodiment, Compound A and darolutamide are dispersed in the same inert carrier. In one embodiment, Compound A and darolutamide are dispersed in the same polymeric carrier.

In one embodiment of the pharmaceutical combination of the present disclosure, Compound A and darolutamide are in separate solid dispersion formulations. In one embodiment, the solid dispersion formulation comprising Compound A and the solid dispersion formulation comprising darolutamide uses different inert carriers. In one embodiment, the solid dispersion formulation comprising Compound A and the solid dispersion formulation comprising darolutamide uses different polymeric carriers. In one embodiment, the solid dispersion formulation comprising Compound A and the solid dispersion formulation comprising darolutamide uses the same polymeric carrier but formulated separately. In one embodiment, the solid dispersion formulation comprising Compound A and the solid dispersion formulation comprising darolutamide are provided in the same dosage form.

In one embodiment of the pharmaceutical combination of the present disclosure, darolutamide is amorphous or crystalline. In one embodiment of the pharmaceutical combination of the present disclosure, darolutamide is crystalline powder.

In one embodiment of the pharmaceutical combination of the present disclosure, Compound A and darolutamide are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical combination of Compound A and darolutamide of the present disclosure, at least one composition is a tablet. In one embodiment, Compound A and darolutamide are in different layers or compartments of the same tablet composition. In one embodiment, the different layers or compartments comprising Compound A and darolutamide are adjacent to one another. In one embodiment, the different layers or compartments comprising Compound A and darolutamide are separated by one or more coatings or compartments such that the two therapeutically active agents do not come in contact. The one or more coatings or compartments separating the different therapeutically active agents can be functional (e.g., modifies release) or inert (e.g., just providing physical separation). In one embodiment, the Compound A layer or the Compound A compartment is about 10% to about 70% by weight of the composition, or about 20% to about 50% by weight of the composition, or about 30% to about 40% by weight of the composition, or any values or subranges therebetween. In one embodiment, the Compound A layer or the Compound A compartment is about 10%, about 15%, about 20%, about 25%, about 30%, about 35%, about 40%, about 45%, about 50%, about 55%, about 60%, about 65%, or about 70% weight of the composition, or any values therebetween. In one embodiment, the layer or the compartment of the composition comprising darolutamide is about 10% to about 70% by weight of the composition or about 20% to about 50% by weight of the composition, or about 30% to about 40% by weight of the composition, or any values or subranges therebetween. In one embodiment, the layer or the compartment of the composition comprising darolutamide is about 10%, about 15%, about 20%, about 25%, about 30%, about 35%, about 40%, about 45%, about 50%, about 55%, about 60%, about 65%, or about 70% by weight of the composition, or any values therebetween.

In one embodiment of the pharmaceutical combination of Compound A and darolutamide of the present disclosure, the combination comprises a third therapeutically active agent. In one embodiment, the third therapeutically active agent is abiraterone or a pharmaceutically acceptable salt, solvate, or prodrug thereof.

In one embodiment of the pharmaceutical combination of Compound A and darolutamide of the present disclosure, the combination comprises a third therapeutically active agent and a fourth therapeutically active agent. In one embodiment, the third therapeutically active agent is abiraterone or a pharmaceutically acceptable salt, solvate, or prodrug thereof, and the fourth therapeutically active agent is any one of the steroids as disclosed herein. In one embodiment, the fourth therapeutically active agent is prednisone, prednisolone, or methylprednisolone.

In one embodiment, the third therapeutically active agent is a CYP3A4 inhibitor. In one embodiment, the CYP3A4 inhibitor is arithromycin, telithromycin, erythromycin, nefazodone, atazanavir, darunavir, indinavir, lopinavir, nelfinavir, saquinavir, tipranavir, ritonavir, ketoconazole, itraconazole, fluconazole, verapamil, or cobicistat, or a pharmaceutically acceptable salt or solvate thereof.

### Compound A+Apalutamide

In one embodiment of the pharmaceutical combination of the present disclosure, the second therapeutically active agent is apalutamide and a daily dosage amount of apalutamide is about 50 mg to about 500 mg, or about 100 mg to about 400 mg, or about 150 mg to about 300 mg, or about 175 mg to about 250 mg, or any values or subranges therebetween. In one embodiment, the daily dosage amount of apalutamide is about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, or about 550 mg, or any values therebetween. In one embodiment, the daily dose of apalutamide is administered once a day, or divided into twice-a-day or three times a day doses. In one embodiment, the daily dose of apalutamide is provided in one tablet or one capsule, or the daily dose is divided into two, three, four, five, or six tablets or capsules.

In one embodiment of the pharmaceutical combination of the present disclosure, the second therapeutically active agent is apalutamide and an amount of apalutamide per a dosage form is about 5 mg to about 100 mg, or about 10 mg to about 75 mg, or about 20 mg to about 60 mg, or about 25 mg to about 50 mg, or any values or subranges therebetween. In one embodiment, the amount of apalutamide per a dosage form is about 5 mg, about 10 mg, about 15 mg, about 20 mg, about 30 mg, about 40 mg, about 50 mg, about 60 mg, about 70 mg, about 80 mg, about 90 mg, or about 100 mg, or any values or subranges therebetween.

In one embodiment of the pharmaceutical combination of the present disclosure, the second therapeutically active agent is apalutamide and an amount of apalutamide per one tablet or one capsule form is about 5 mg to about 100 mg, or about 10 mg to about 75 mg, or about 20 mg to about 60 mg, or about 25 mg to about 50 mg, or any values or subranges therebetween. In one embodiment, the amount of apalutamide per one tablet or one capsule is about 5 mg, about 10 mg, about 15 mg, about 20 mg, about 30 mg, about 40 mg, about 50 mg, about 60 mg, about 70 mg, about 80 mg, about 90 mg, or about 100 mg, or any values or subranges therebetween. In one embodiment, the amount of apalutamide per one tablet or one capsule is about 60 mg.

In one embodiment of the pharmaceutical combination of Compound A and apalutamide of the present disclosure, Compound A and apalutamide are in the same composition. In one embodiment, Compound A and apalutamide are in different compositions. In one embodiment, at least one composition comprises a solid dispersion formulation. In one embodiment, at least one composition in the combination comprises hydroxypropyl methylcellulose acetate succinate.

In one embodiment, the composition comprising apalutamide comprises one or more pharmaceutically acceptable carrier or excipient selected from colloidal anhydrous silica, croscarmellose sodium, hydroxypropyl methylcellulose-acetate succinate, magnesium stearate, microcrystalline cellulose, or silicified microcrystalline cellulose. In one embodiment, the composition comprising apalutamide is a tablet. In one embodiment, the tablet comprising apalutamide has a film-coat. In one embodiment, the film-coat comprises one or more pharmaceutically acceptable carrier or excipient selected from iron oxide black, iron oxide yellow, polyethylene glycol, polyvinyl alcohol, talc, or titanium dioxide. Examples of formulations of apalutamide are disclosed in WO2017/209939 A1 and WO2018/112001 A1, the disclosures of which are hereby incorporated by reference in their entirety.

In one embodiment of the pharmaceutical combination of the present disclosure, Compound A and apalutamide are in the same solid dispersion formulation. In one embodiment, Compound A and apalutamide are dispersed in the same inert carrier. In one embodiment, Compound A and apalutamide are dispersed in the same polymeric carrier.

In one embodiment of the pharmaceutical combination of the present disclosure, Compound A and apalutamide are in separate solid dispersion formulations. In one embodiment, the solid dispersion formulation comprising Compound A and the solid dispersion formulation comprising apalutamide uses different inert carriers. In one embodiment, the solid dispersion formulation comprising Compound A and the solid dispersion formulation comprising apalutamide uses different polymeric carriers. In one embodiment, the solid dispersion formulation comprising Compound A and the solid dispersion formulation comprising apalutamide uses the same polymeric carrier but formulated separately. In one embodiment, the solid dispersion formulation comprising Compound A and the solid dispersion formulation comprising apalutamide are provided in the same dosage form.

In one embodiment of the pharmaceutical combination of the present disclosure, apalutamide is amorphous or crystalline. In one embodiment of the pharmaceutical combination of the present disclosure, apalutamide is amorphous.

In one embodiment of the pharmaceutical combination of the present disclosure, Compound A and apalutamide are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical combination of Compound A and apalutamide of the present disclosure, at least one composition is a tablet. In one embodiment, Compound A and apalutamide are in different layers or compartments of the same tablet composition. In one embodiment, the different layers or compartments comprising Compound A and apalutamide are adjacent to one another. In one embodiment, the different layers or compartments comprising Compound A and apalutamide are separated by one or more coatings or compartments such that the two therapeutically active agents do not come in contact. The one or more coatings or compartments separating the different therapeutically active agents can be functional (e.g., modifies release) or inert (e.g., just providing physical separation). In one embodiment, the Compound A layer or the Compound A compartment is about 10% to about 70% by weight of the composition, or about 20% to about 50% by weight of the composition, or about 30% to about 40% by weight of the composition, or any values or subranges therebetween. In one embodiment, the Compound A layer or the Compound A compartment is about 10%, about 15%, about 20%, about 25%, about 30%, about 35%, about 40%, about 45%, about 50%, about 55%, about 60%, about 65%, or about 70% weight of the composition, or any values therebetween. In one embodiment, the layer or the compartment of the composition comprising apalutamide is about 10% to about 70% by weight of the composition or about 20% to about 50% by weight of the composition, or about 30% to about 40% by weight of the composition, or any values or subranges therebetween. In one embodiment, the layer or the compartment of the composition comprising apalutamide is about 10%, about 15%, about 20%, about 25%, about 30%, about 35%, about 40%, about 45%, about 50%, about 55%, about 60%, about 65%, or about 70% by weight of the composition, or any values therebetween.

In one embodiment of the pharmaceutical combination of Compound A and apalutamide of the present disclosure, the combination comprises a third therapeutically active agent. In one embodiment, the third therapeutically active agent is abiraterone or a pharmaceutically acceptable salt, solvate, or prodrug thereof.

In one embodiment of the pharmaceutical combination of Compound A and apalutamide of the present disclosure, the combination comprises a third therapeutically active agent and a fourth therapeutically active agent. In one embodiment, the third therapeutically active agent is abiraterone or a pharmaceutically acceptable salt, solvate, or prodrug thereof, and the fourth therapeutically active agent is any one of the steroids as disclosed herein. In one embodiment, the fourth therapeutically active agent is prednisone, prednisolone, or methylprednisolone.

In one embodiment, the third therapeutically active agent is a CYP3A4 inhibitor.

### Compound A+Abiraterone

In one embodiment of the pharmaceutical combination of the present disclosure, the second therapeutically active agent is abiraterone or a pharmaceutically acceptable salt, solvate, or prodrug thereof. In one embodiment, the second therapeutically active agent is abiraterone. In one embodiment, the second therapeutically active agent is abiraterone acetate.

In one embodiment of the pharmaceutical combination of the present disclosure, a daily dosage amount of abiraterone or a pharmaceutically acceptable salt, solvate, or prodrug thereof is about 100 mg to about 2,000 mg, or about 200 mg to about 1,000 mg, or about 300 mg to about 800 mg, or about 400 mg to about 600 mg, or any values or subranges therebetween. In one embodiment, the daily dosage amount of abiraterone or a pharmaceutically acceptable salt, solvate, or prodrug thereof is about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg, or about 2,000 mg, or any values therebetween. In one embodiment, the daily dose of abiraterone or a pharmaceutically acceptable salt, solvate, or prodrug thereof is administered once a day, or divided into twice-a-day or three times a day doses. In one embodiment, the daily dose of abiraterone or a pharmaceutically acceptable salt, solvate, or prodrug thereof is provided in one tablet or one capsule, or the daily dose is divided into two, three, four, five, or six tablets or capsules.

In one embodiment of the pharmaceutical combination of the present disclosure, the second therapeutically active agent is abiraterone or a pharmaceutically acceptable salt, solvate, or prodrug thereof and an amount of abiraterone or a pharmaceutically acceptable salt, solvate, or prodrug thereof per a dosage form is about 50 mg to about 1,000 mg, or about 75 mg to about 800 mg, or about 100 mg to about 600 mg, or about 125 mg to about 500 mg, or any values or subranges therebetween. In one embodiment, the amount of abiraterone or a pharmaceutically acceptable salt, solvate, or prodrug thereof per a dosage form is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values or subranges therebetween.

In one embodiment of the pharmaceutical combination of the present disclosure, the second therapeutically active agent is abiraterone or a pharmaceutically acceptable salt, solvate, or prodrug thereof and an amount of abiraterone or a pharmaceutically acceptable salt, solvate, or prodrug thereof per one tablet or one capsule form is about 50 mg to about 1,000 mg, or about 75 mg to about 800 mg, or about 100 mg to about 600 mg, or about 125 mg to about 500 mg, or any values or subranges therebetween. In one embodiment, the amount of abiraterone or a pharmaceutically acceptable salt, solvate, or prodrug thereof per one tablet or one capsule is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, or any values or subranges therebetween. In one embodiment, the amount of abiraterone or a pharmaceutically acceptable salt, solvate, or prodrug thereof per one tablet or one capsule is about 125 mg, about 250 mg, or about 500 mg.

In one embodiment of the pharmaceutical combination of Compound A and abiraterone or a pharmaceutically acceptable salt, solvate, or prodrug thereof of the present disclosure, Compound A and abiraterone or a pharmaceutically acceptable salt, solvate, or prodrug thereof are in the same composition. In one embodiment, Compound A and abiraterone or a pharmaceutically acceptable salt, solvate, or prodrug thereof are in different compositions. In one embodiment, at least one composition comprises a solid dispersion formulation.

In one embodiment, the composition comprising abiraterone or a pharmaceutically acceptable salt, solvate, or prodrug hereof comprises one or more pharmaceutically acceptable carrier or excipient selected from lactose monohydrate, microcrystalline cellulose, croscarmellose sodium, povidone, sodium lauryl sulfate, magnesium stearate, or colloidal silicon dioxide. In one embodiment, the composition comprising abiraterone or a pharmaceutically acceptable salt, solvate, or prodrug thereof comprises one or more pharmaceutically acceptable carrier or excipient selected from lactose monohydrate, microcrystalline cellulose, croscarmellose sodium, sodium lauryl sulfate, sodium stearyl fumarate, butylated hydroxyanisole, or butylated hydroxytoluene. In one embodiment, the composition comprising abiraterone or a pharmaceutically acceptable salt or solvate thereof is a table. In one embodiment, the tablet comprising abiraterone or a pharmaceutically acceptable salt, solvate, or prodrug thereof has a film-coat. Examples of formulations of abiraterone are disclosed in WO2019/060525 A1, WO2017/209939 A1 and WO2019/03284A1, the disclosures of which are hereby incorporated by reference in their entirety.

In one embodiment of the pharmaceutical combination of the present disclosure, Compound A and abiraterone or a pharmaceutically acceptable salt, solvate, or prodrug thereof are in the same solid dispersion formulation. In one embodiment, Compound A and abiraterone or a pharmaceutically acceptable salt, solvate, or prodrug thereof are dispersed in the same inert carrier. In one embodiment, Compound A and abiraterone or a pharmaceutically acceptable salt, solvate, or prodrug thereof are dispersed in the same polymeric carrier.

In one embodiment of the pharmaceutical combination of the present disclosure, Compound A and abiraterone or a pharmaceutically acceptable salt, solvate, or prodrug thereof are in separate solid dispersion formulations. In one embodiment, the solid dispersion formulation comprising Compound A and the solid dispersion formulation comprising abiraterone or a pharmaceutically acceptable salt, solvate, or prodrug thereof uses different inert carriers. In one embodiment, the solid dispersion formulation comprising Compound A and the solid dispersion formulation comprising abiraterone or a pharmaceutically acceptable salt, solvate, or prodrug thereof uses different polymeric carriers. In one embodiment, the solid dispersion formulation comprising Compound A and the solid dispersion formulation comprising abiraterone or a pharmaceutically acceptable salt, solvate, or prodrug thereof uses the same polymeric carrier but formulated separately. In one embodiment, the solid dispersion formulation comprising Compound A and the solid dispersion formulation comprising abiraterone or a pharmaceutically acceptable salt, solvate, or prodrug thereof are provided in the same dosage form.

In one embodiment of the pharmaceutical combination of the present disclosure, abiraterone or a pharmaceutically acceptable salt, solvate, or prodrug thereof is amorphous or crystalline. In one embodiment of the pharmaceutical combination of the present disclosure, abiraterone or a pharmaceutically acceptable salt, solvate, or prodrug thereof is crystalline powder.

In one embodiment of the pharmaceutical combination of the present disclosure, Compound A and abiraterone or a pharmaceutically acceptable salt, solvate, or prodrug thereof are in different compositions but provided in a single kit. In one embodiment, the kit comprises 1, 2, 3, 4, 5, or 6 compositions for each therapeutically active agent to be administered per day. In one embodiment, the kit comprises 1, 2, 3, 4, 5, or 6 tablets or capsules or mixtures of tablets and capsules for each therapeutically active agent.

In one embodiment of the pharmaceutical combination of Compound A and abiraterone or a pharmaceutically acceptable salt, solvate, or prodrug thereof of the present disclosure, at least one composition is a tablet. In one embodiment, Compound A and abiraterone or a pharmaceutically acceptable salt, solvate, or prodrug thereof are in different layers or compartments of the same tablet composition. In one embodiment, the different layers or compartments comprising Compound A and abiraterone or a pharmaceutically acceptable salt, solvate, or prodrug thereof are adjacent to one another. In one embodiment, the different layers or compartments comprising Compound A and abiraterone or a pharmaceutically acceptable salt, solvate, or prodrug thereof are separated by one or more coatings or compartments such that the two therapeutically active agents do not come in contact. The one or more coatings or compartments separating the different therapeutically active agents can be functional (e.g., modifies release) or inert (e.g., just providing physical separation). In one embodiment, the Compound A layer or the Compound A compartment is about 10% to about 70% by weight of the composition, or about 20% to about 50% by weight of the composition, or about 30% to about 40% by weight of the composition, or any values or subranges therebetween. In one embodiment, the Compound A layer or the Compound A compartment is about 10%, about 15%, about 20%, about 25%, about 30%, about 35%, about 40%, about 45%, about 50%, about 55%, about 60%, about 65%, or about 70% weight of the composition, or any values therebetween. In one embodiment, the layer or the compartment of the composition comprising abiraterone or a pharmaceutically acceptable salt, solvate, or prodrug thereof is about 10% to about 70% by weight of the composition or about 20% to about 50% by weight of the composition, or about 30% to about 40% by weight of the composition, or any values or subranges therebetween. In one embodiment, the layer or the compartment of the composition comprising abiraterone or a pharmaceutically acceptable salt, solvate, or prodrug thereof is about 10%, about 15%, about 20%, about 25%, about 30%, about 35%, about 40%, about 45%, about 50%, about 55%, about 60%, about 65%, or about 70% by weight of the composition, or any values therebetween.

In one embodiment of the pharmaceutical combination of Compound A and abiraterone or a pharmaceutically acceptable salt, solvate, or prodrug thereof of the present disclosure, the combination comprises a third therapeutically active agent. In one embodiment, the third therapeutically active agent is an AR LBD inhibitor. In one embodiment, the third therapeutically active agent is enzalutamide, darolutamide, or apalutamide. In one embodiment, the third therapeutically active agent is a CYP3A4 inhibitor. In one embodiment, the third therapeutically active agent is any one of the steroids as disclosed herein. In one embodiment, the third therapeutically active agent is prednisone, prednisolone, or methylprednisolone.

In one embodiment of the pharmaceutical combination of the present disclosure, the third therapeutically active agent is a steroid and a daily dosage amount of the steroid is about 1 mg to about 50 mg, or about 2 mg to about 40 mg, or about 3 mg to about 30 mg, or about 5 mg to about 20 mg, or any values or subranges therebetween. In one embodiment, the daily dosage amount of steroid is about 1 mg, about 2 mg, about 3 mg, about 4 mg, about 5 mg, about 6 mg, about 7 mg, about 8 mg, about 9 mg, about 10 mg, about 12 mg, about 14 mg, about 16 mg, about 18 mg, about 20 mg, or about 25 mg, about 30 mg, about 35 mg, about 40 mg, about 45 mg, or about 50 mg, or any values therebetween. In one embodiment, the daily dose of steroid is administered once a day, or divided into twice-a-day or three times a day doses. In one embodiment, the daily dose of steroid is provided in one tablet or one capsule, or the daily dose is divided into two, three, four, five, or six tablets or capsules.

In one embodiment of the pharmaceutical combination of the present disclosure, the third therapeutically active agent is a steroid and an amount of the steroid per a dosage form is about 1 mg to about 30 mg, or about 2 mg to about 25 mg, or about 3 mg to about 15 mg, or about 4 mg to about 10 mg, or any values or subranges therebetween. In one embodiment, the amount of steroid per a dosage form is about 1 mg, about 2 mg, about 3 mg, about 4 mg, about 5 mg, about 6 mg, about 7 mg, about 8 mg, about 9 mg, about 10 mg, about 12 mg, about 14 mg, about 16 mg, about 18 mg, about 20 mg, or about 25 mg, about 30 mg, or any values or subranges therebetween.

In one embodiment of the pharmaceutical combination of the present disclosure, the third therapeutically active agent is a steroid and an amount of the steroid per one tablet or one capsule form is about 1 mg to about 30 mg, or about 2 mg to about 25 mg, or about 3 mg to about 15 mg, or about 4 mg to about 10 mg, or any values or subranges therebetween. In one embodiment, the amount of apalutamide per one tablet or one capsule is about 1 mg, about 2 mg, about 3 mg, about 4 mg, about 5 mg, about 6 mg, about 7 mg, about 8 mg, about 9 mg, about 10 mg, about 12 mg, about 14 mg, about 16 mg, about 18 mg, about 20 mg, or about 25 mg, about 30 mg, or any values or subranges therebetween. In one embodiment, the third therapeutically active agent is prednisone, prednisolone, or methylprednisolone. In one embodiment, prednisone, prednisolone, or methylprednisolone per one tablet or one capsule is about 4 mg or about 5 mg.

In one embodiment of the pharmaceutical combination of Compound A and abiraterone or a pharmaceutically acceptable salt, solvate, or prodrug thereof, and a steroid of the present disclosure, the combination comprises a fourth therapeutically active agent. In one embodiment, the fourth therapeutically active agent is an AR LBD inhibitor. In one embodiment, the fourth therapeutically active agent is enzalutamide, darolutamide, or apalutamide.

In one embodiment, the fourth therapeutically active agent is a CYP3A4 inhibitor.

### Various Embodiments of Pharmaceutical Compositions, Formulations, Dosage Forms

The pharmaceutical composition or a combination as disclosed herein, can further comprise a pharmaceutically acceptable carrier or excipient.

In a further embodiment of the present disclosure, a pharmaceutical composition or combination as disclosed herein comprises a pharmaceutically acceptable carrier, excipient or adjuvant is provided. The pharmaceutically acceptable carriers, excipients and adjuvants are added to the composition or formulation for a variety of purposes. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection.

Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, PA.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

Additional embodiments relate to the pharmaceutical formulations wherein the formulation is selected from the group consisting of a solid, powder, liquid and a gel. In certain embodiments, a pharmaceutical composition of the present invention is a solid (e.g., a powder, tablet, a capsule, granulates, and/or aggregates). In certain of such embodiments, a solid pharmaceutical composition comprising one or more ingredients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid pharmaceutical compositions can optionally have different types of coating. Coatings can be applied to the entire dosage form (e.g. a tablet) or a component of a dosage form (e.g., core, granules, beads, pellets, microparticles, etc). A coating can be used to improve patient compliance (e.g., taste-masking coating, flavor coating, coating to provide smooth surface for easy swallowing), to improve the stability of the compositions (e.g., protection from light, moisture, gas, acid protection, or to divide different layers or compartments to avoid a drug from interacting with different ingredients in a different layer/compartment), alter release profile of the drug (e.g., enteric coating, pH-dependent polymer coating, etc), or improve cosmetic considerations.

A coating can be a thin film-coating comprising one or more polymers or water soluble materials including but are not limited to, hypromellose, macrogol, hydroxyethylcellulose, sodium carboxymethylcellulose, polyethylene glycol, polyvinyl alcohol, and cellulose acetate phthalate. In addition, film coating can comprise one or more pharmaceutically acceptable excipients, including but not limited to titanium dioxide, ferric oxide, coloring agents, talc, or lecithin.

A coating that modifies release of the active ingredient can comprise a pH-dependent polymer (e.g., enteric polymer) or a pH-independent polymer. A release-modifying coating can comprise one or more polymers selected from methacrylic copolymers, aminoalkyl methacrylate copolymers, methacrylate copolymers, or ammonioalkyl methacrylate copolymers. A release-modifying coating can comprise one or more cationic polymer, anionic polymer, or neutral polymer.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared where the solid or amorphous components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80 and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present disclosure is prepared for oral administration. In certain of such embodiments, a pharmaceutical composition is formulated by combining one or more agents and pharmaceutically acceptable carriers. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In other embodiments the compound of the present disclosure are administered by the intravenous route. In further embodiments, the parenteral administration may be provided in a bolus or by infusion.

In certain embodiments, a pharmaceutical composition is prepared for rectal administration, such as a suppository or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more therapeutically active agents, or a pharmaceutically acceptable salt or solvate thereof are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances, the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In various aspects, the androgen receptor modulators in the pharmaceutical composition or combination as disclosed herein can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be administered daily or more frequently depending upon a number of factors, including the overall health of a patient, and the formulation and route of administration of the selected compound(s). An ordinarily skilled physician or veterinarian can readily determine and prescribe the effective amount of the drug required to prevent, counter or arrest the progress of the condition.

The compounds or pharmaceutical compositions of the present disclosure may be manufactured and/or administered in single or multiple unit dose forms.

Having now generally described the invention, the same will be more readily understood through reference to the following examples, which are provided by way of illustration and are not intended to be limiting of the present invention.

### EXAMPLES

The disclosure now being generally described, it will be more readily understood by reference to the following examples which are included merely for purposes of illustration of certain aspects and embodiments of the present invention, and are not intended to limit the invention.

### Example 1: Synthesis of N-(4-((4-(2-(3-chloro-4-(2-chloroethoxy)-5-cyanophenyl)propan-2-yl)phenoxy)methyl)pyrimidin-2-yl)methanesulfonamideN-(4-((4-(2-(3-chloro-4-(2-chloroethoxy)-5-cyanophenyl) propan-2-yl) phenoxy)methyl)pyrimidin-2-yl)methanesulfonamide (A)

2-chloro-4-(chloromethyl)pyrimidine: To a mixture of 2-chloro-4-methyl-pyrimidine (50.0 g, 398 mmol) and NCS (77.9 g, 583 mmol) in MeCN (250 mL) was added benzoyl benzenecarboperoxoate (28.3 g, 117 mmol) in portions at 20° C. and the mixture was stirred at 100° C. for 16 hrs under N₂ atmosphere. TLC showed most of the starting material consumed and two new spots appeared. The mixture was cooled down to room temperature, poured into water (500 mL) and extracted with EtOAc (200 mL×3). The organic layers were combined and washed with brine (200 mL×2), dried over Na₂SO₄, filtered and concentrated under reduced pressure. The residue was purified by silica gel column chromatography to give 2-chloro-4-(chloromethyl)pyrimidine (22 g, yield: 31.2%) as yellow oil. ¹H NMR (400 MHz, CDCl3) δ=8.69 (d, J=5.2 Hz, 1H), 7.54 (d, J=5.0 Hz, 1H), 4.61 (s, 2H).

3-chloro-2-(2-chloroethoxy)-5-(2-(4-((2-chloropyrimidin-4-yl)methoxy)phenyl)propan-2-yl)benzonitrile: To a mixture of 3-chloro-2-(2-chloroethoxy)-5-(2-(4-hydroxyphenyl)propan-2-yl)benzonitrile (18.0 g, 51.4 mmol) and 2-chloro-4-(chloromethyl)pyrimidine (10.1 g, 61.7 mmol) in DMF (150 mL) was added Cs₂CO₃ (33.5 g, 103.4 mmol) at 20° C. and the mixture was stirred at the same temperature for 16 hrs. LCMS showed the reaction was completed. The reaction mixture was poured into H₂O (300 mL) and extracted with EtOAc (150 mL×3). The combined organic layers were washed with brine (150 mL×3), dried over Na₂SO₄, filtered and concentrated under reduced pressure. The residue was purified by silica gel column chromatography to give 3-chloro-2-(2-chloroethoxy)-5-(2-(4-((2-chloropyrimidin-4-yl)methoxy)phenyl)propan-2-yl)benzonitrile (15.5 g, yield: 63.3%) as white solid. ¹H NMR (400 MHz, CDCl3) δ=8.67 (d, J=5.2 Hz, 1H), 7.56 (d, J=5.2 Hz, 1H), 7.45 (d, J=2.4 Hz, 1H), 7.35-7.29 (m, 1H), 7.13 (d, J=8.8 Hz, 2H), 6.90 (d, J=8.8 Hz, 2H), 5.16 (s, 2H), 4.43 (t, J=6.0 Hz, 2H), 3.88 (t, J=6.0 Hz, 2H), 1.65 (s, 6H).

N-(4-((4-(2-(3-chloro-4-(2-chloroethoxy)-5-cyanophenyl)propan-2-yl)phenoxy)methyl)pyrimidin-2-yl)methanesulfonamide (A): To a mixture of 3-chloro-2-(2-chloroethoxy)-5-(2-(4-((2-chloropyrimidin-4-yl)methoxy)phenyl)propan-2-yl)benzonitrile (15.5 g, 32.5 mmol), methane sulfonamide (9.3 g, 97.5 mmol), Cs₂CO₃ (21.2 g, 65.0 mmol) and Xantphos (1.88 g, 3.25 mmol) in 1,4-dioxane (450 mL) was added Pd₂(dba)₃ (3.0 g, 3.3 mmol) at 20° C. and the mixture was stirred at 90° C. for 6 hrs under N₂ atmosphere. LCMS showed the reaction was completed. The mixture was cooled down to room temperature, poured into water (300 mL) and extracted with EtOAc (300 mL×3). The combined organic layers were washed with brine (300 mL×2), dried over Na₂SO₄, filtered and concentrated under reduced pressure. The residue was purified by silica gel column chromatography to give the crude product and then further purified by p-HPLC (TFA) to give N-(4-((4-(2-(3-chloro-4-(2-chloroethoxy)-5-cyanophenyl)propan-2-yl)phenoxy)methyl)pyrimidin-2-yl)methanesulfonamide (5.30 g, yield: 30.1%) as yellow solid. ¹H NMR (400 MHz, CDCl3) δ=10.02 (br s, 1H), 8.69 (d, J=5.2 Hz, 1H), 7.45 (d, J=2.4 Hz, 1H), 7.34-7.31 (m, 1H), 7.30 (d, J=5.2 Hz, 1H), 7.13 (d, J=8.8 Hz, 2H), 6.91 (d, J=8.8 Hz, 2H), 5.13 (s, 2H), 4.43 (t, J=6.0 Hz, 2H), 3.88 (t, J=6.0 Hz, 2H), 3.47 (s, 3H), 1.65 (s, 6H). LCMS (220 nm): 99.0%. Exact Mass: 534.09; found 535.1, 537.0. See WO 2020/081999.

### Biological Assays

### Example 2: Activity of Exemplary Compounds in Cellular Assays

LNCaP cells were transiently transfected with the PSA (6.1 kb)-luciferase reporter for 24 h, and then treated with indicated concentration of representative compounds with synthetic androgen, R1881 (1 nM) for 24 h. After 24 h of incubation with R1881, the cells were harvested, and relative luciferase activities were determined. To determine the IC₅₀, treatments were normalized to the maximum activity with androgen-induction (in the absence of test compounds, vehicle only) (Table 1).

Luciferase Assay: Lysates were thawed on ice then collected into V-bottom 96-well tissue culture plates. Lysates were centrifuged at 4° C. for 5 minutes at 4000 rpm. To measure luminescence of LNCaP cell lysates the Firefly Luciferase Assay System (Promega) was employed, according to manufacturer's protocol.

Statistical analyses were performed using GraphPad Prism (Version 6.01 for Windows; La Jolla, CA, USA). Comparisons between treatment and control groups were compared using Two-Way ANOVA with post-hoc Dunnett's and Tukey's tests. Differences were considered statistically significant at P values less than 0.05. Densitometric quantification of relative AR levels was determined by Image.

Table 1 shows the IC₅₀ of representative Compounds from Tables A-B from androgen-induced PSA luciferase assay. EPI-002 has the following structure:

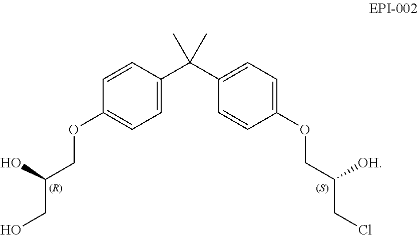

TABLE 1
IC₅₀ of Compound A on Androgen-Induced
PSA Luciferase Activity

| | Androgen-induced | |
|---|---|---|
| Compound | PSA-luciferase | |
| ID | IC₅₀ (nM) | n |
| A | 535 | 2 |
| EPI-002 | 9580 | 2 |
| Enzalutamide | 189 | 8 |
| Bicalutamide | 306 | 2 |

### Example 3. In Vivo Pharmacokinetic Properties

The purpose of this study is to determine the pharmacokinetics of the tested compounds in plasma, following oral gavage administration (PO) to male CD-1 mice.

Animal Husbandry: Animals were group housed during acclimation and the study. The animal room environment were controlled (target conditions: temperature 18 to 26° C., relative humidity 30 to 70%, 12 hours artificial light and 12 hours dark). Temperature and relative humidity were monitored daily. Animals were fasted at least 12 hours prior to the administration. All animals had access to Certified Rodent Diet ad libitum 4 hours post dosing.

Dose Formulation: prepared 85-100 μL of a stock solution of representative compounds of the disclosure in DMSO at 50 mM; 1.5% v/v of Tween 80 were added and mixed gently; 95.5% v/v saline was added gently to the organic phase. The solution was mixed slowly by reverse pipetting to get a clear solution.

Dose Administration: the dose formulations were administered via oral gavage per facility SOPs. The dose volume were determined by the animals' body weight collected on the morning of dosing day

Sample Collection: blood collection (about 0.05 mL per time point) was performed from saphenous vein of each animal into polypropylene tubes at each timepoints (0, 0.25, 0.5, 1, 2, 4, 8, 12, and 24 hr). All blood samples were transferred into pre-chilled tubes containing 2 μL of K2-EDTA (0.5M) and placed on wet ice until centrifugation. Each collected blood sample was in the wet-ice before centrifugation. Each collected blood was under centrifugation for 15 minutes at 4° C. and 3000 g for plasma collection. Plasma samples were stored in polypropylene tubes, quickly frozen over dry ice and kept at −70±10° C. until LC/MS/MS analysis.

Bioanalytical analysis: A LC-MS/MS method for the quantitative determination of tested compound in biological matrix were developed under non-GLP compliance. A calibration curve with at least 7 non-zero calibration standards were applied for the method including LLOQ.

Plasma concentration versus time data were analyzed by non-compartmental approaches using the Phoenix WinNonlin 6.3 software program. Cl, Vdss, C0, Cmax, Tmax, T½, AUC(0-t), AUC(0-inf), MRT(0-t), MRT(0-inf), % F (oral availability) and graphs of plasma concentration versus time profile were reported.

Tables 2 shows PK parameters determined from single PO dose of Compound A and enzalutamide, dosed as single agent or combination in CD-1 male mice.

TABLE 2
PK Parameters (PO)

| Compound | Dose | Cmax | tmax | T½ | AUC₀₋ₗₐₛₜ |
|---|---|---|---|---|---|
| ID | (mg/kg) | (ng/mL) | (hr) | (hr) | (hr*ng/mL) |
| A | 60.00 | 41,667 | 4 | 5.4 | 482,822 |
| Enzalutamide | 15.0 | 21,067 | 9 | ND | 388,202 |
| A + Enzalutamide | 60.00 | 51,900 | 3 | 6.9 | 603,960 |
| Enzalutamide + A | 15.0 | 11,467 | 12 | ND | 231,974 |

### Example 4. In Vivo Activity of Compound a in VCaP Xenografts Model

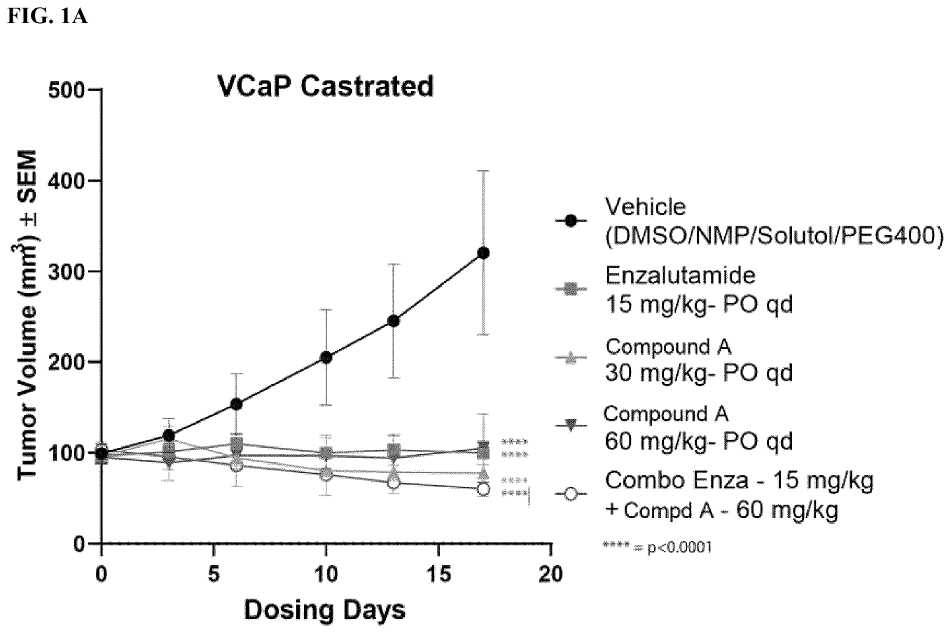

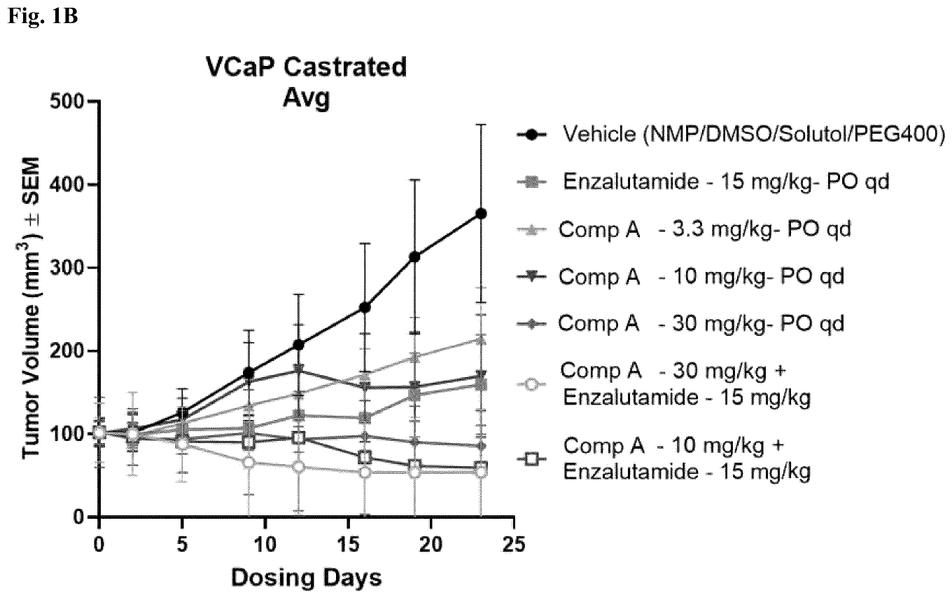

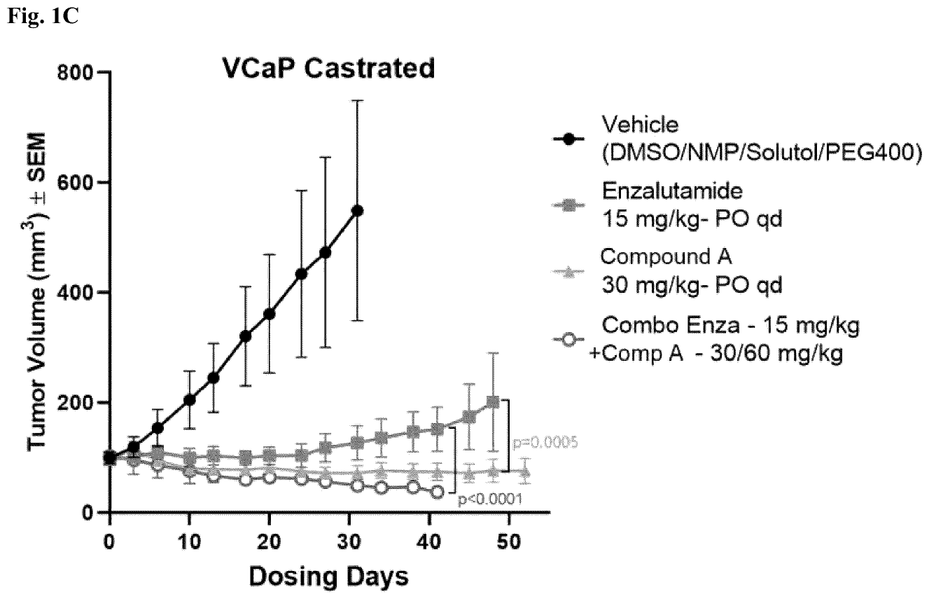

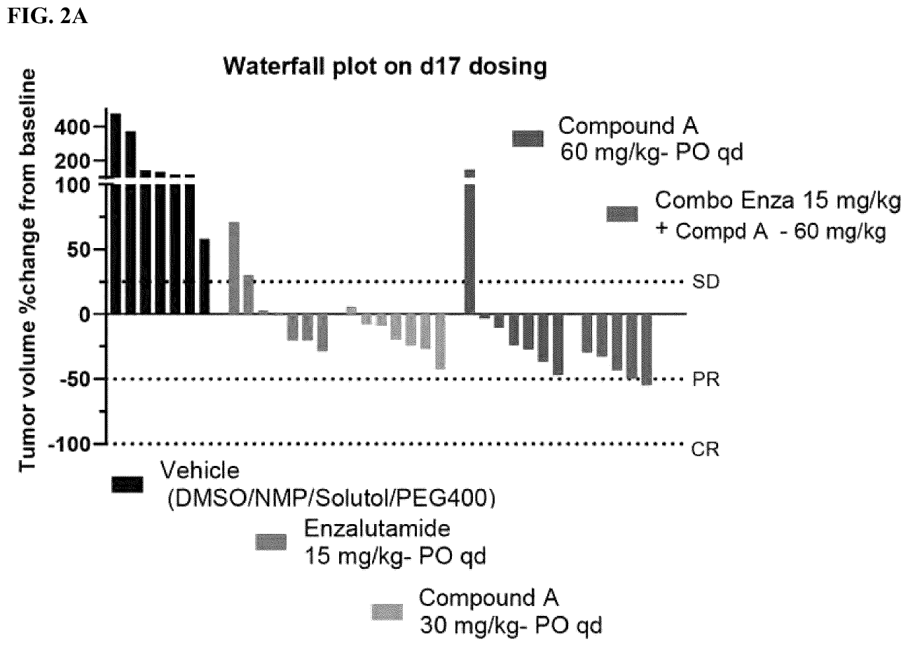

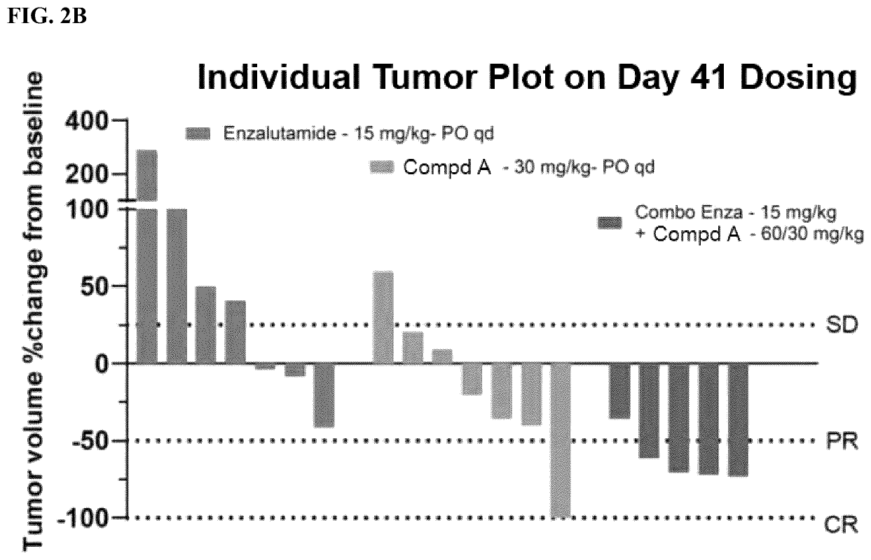

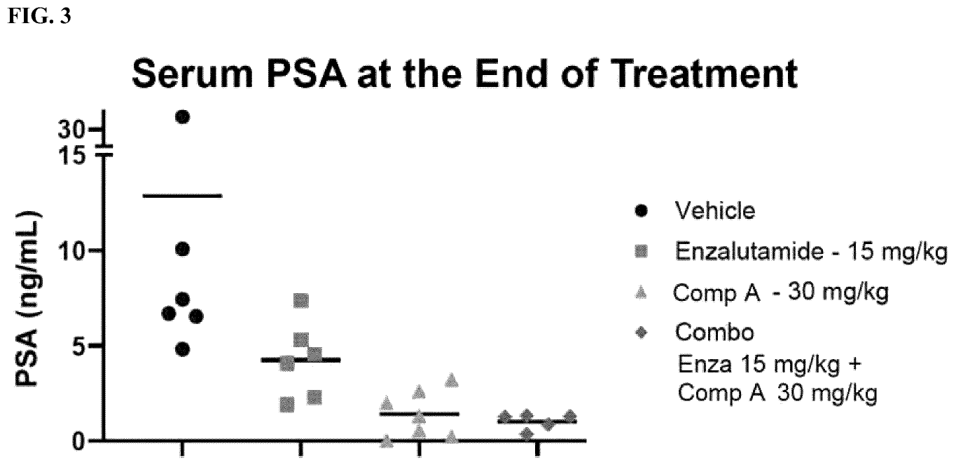

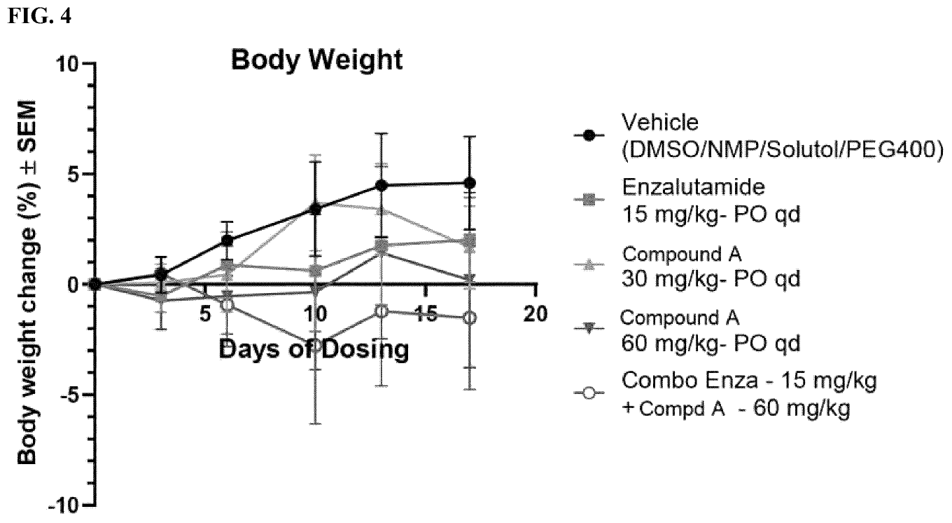

Tumor growth was measured in male SCID Beige mice bearing VCaP tumors. Castration was performed when tumors reached ˜ 100 mm³ and dosing (as indicated) of enzalutamide, Compound A, or combination of enzalutamide and Compound A started 2 weeks after castration (FIGS. 1A-1C). Individual tumor volume change from baseline measured on day 17 of the experiment (FIGS. 2A-2B). Serum prostate-specific antigen (PSA) was measured in mice at the end of each treatment (FIG. 3). Body weight of the mice were captured biweekly in the animals and normalized to baseline (FIG. 4). Data demonstrated in FIGS. 1-4 shows that Compound A exhibits excellent anti-tumor response as a monotherapy or in combination with enzalutamide.

### Example 5. Inhibition of Androgen Responsive GENES in LNCaP Cells

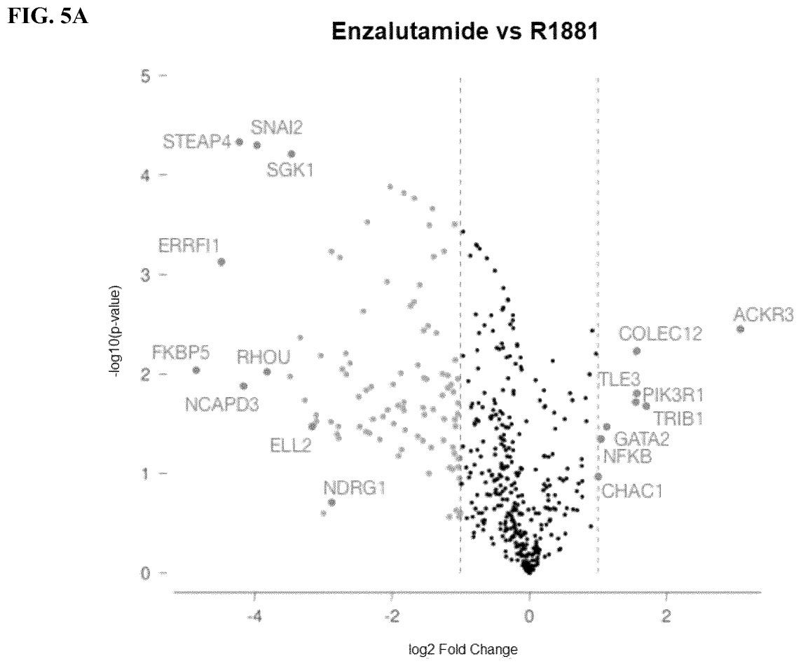

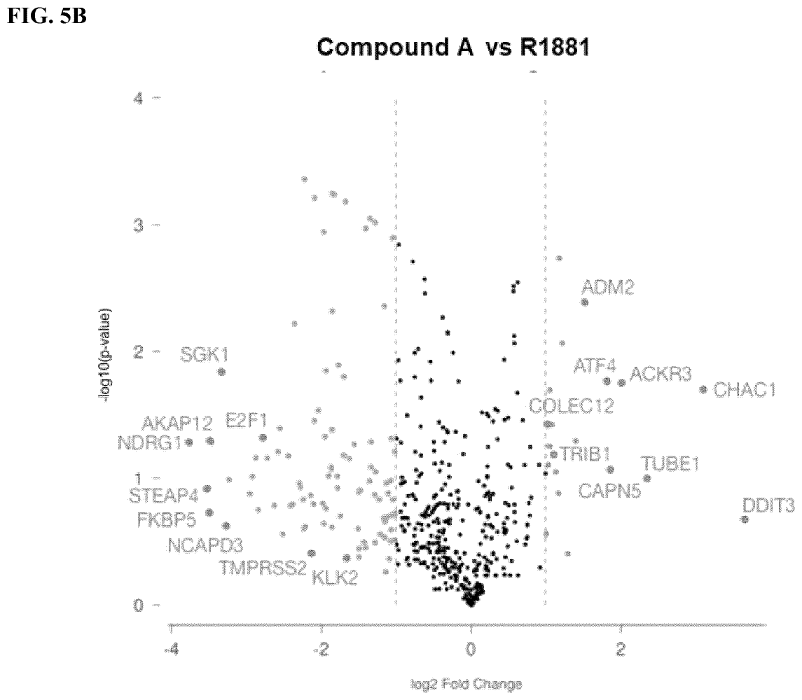

Relative mRNA expression in LNCaP was measured to assess dose-dependent response of Compound A alone or with enzalutamide (Enza), apalutamide (Apa), or darolutamide (Daro) in androgen responsive genes. FIGS. 5A and 5B show log-ratio of gene expression values in LNCaP cells treated with enzalutamide compared to synthetic androgen (R1881) and LNCaP cells treated with Compound A compared to R1881, respectively.

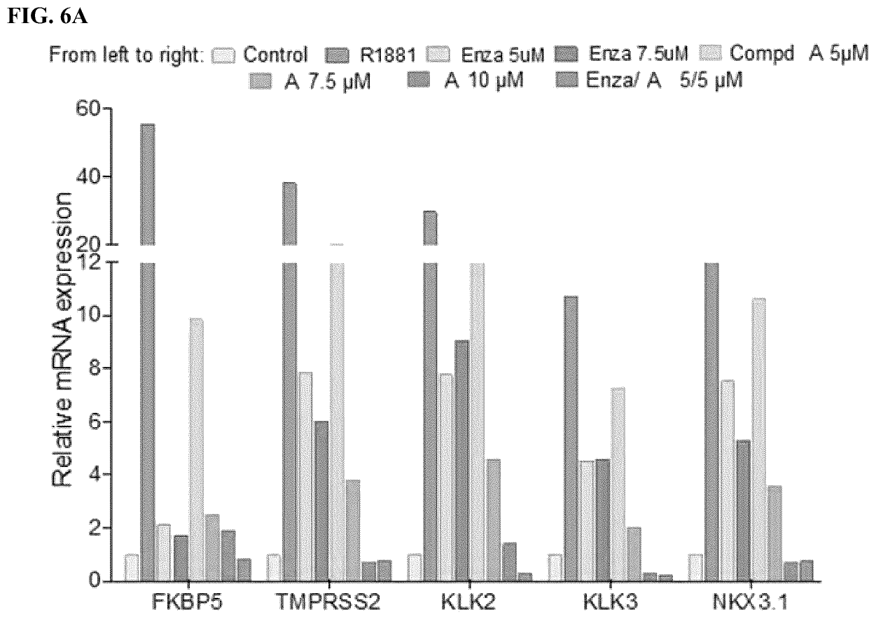

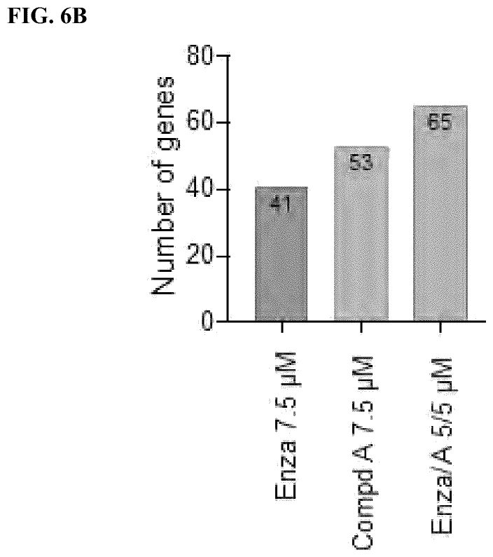

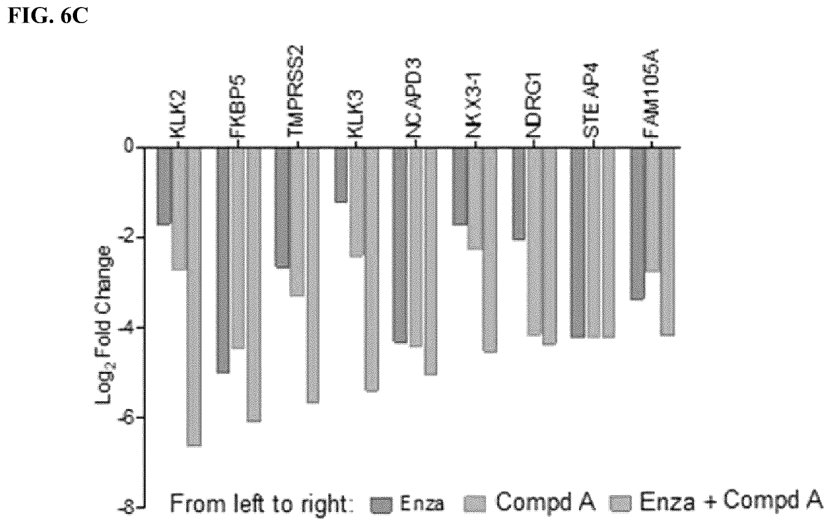

Compound A demonstrated dose-dependent response in the androgen response genes FKBP5, TMPRSS2, KLK2, KLK3, and NKX3.1 (FIG. 6A). The combination of enzalutamide and Compound A exhibited inhibition of the tested androgen responsive genes.

The number of genes significantly down-regulated with Fold changes ≥4 is shown in FIG. 6B. Top ten down-regulated genes with Enza/Compound A 5 μM/5 μM combination is shown in FIG. 6C. Top twenty down-regulated genes with Enza/Compound A 5 μM/5 μM combination is shown in Table 4. FIGS. 5A-5B and Table 4 demonstrate that Compound A can modulate androgen receptor0 full-length driven genes similar to enzalutamide in LNCaP.

TABLE 4
Top 20 down-regulated genes with Enza
and Compound A, alone or in combination

| | | Log2Fold Change | | |
|---|---|---|---|---|
| | | 7.5 μM | 7.5 μM | 5/5 μM |
| | Gene | Enza | Compound A | Combo |
| 1. | KLK2 | −1.72 | −2.71 | −6.63 |
| 2. | FKBP5 | −5.01 | −4.47 | −6.09 |
| 3. | TMPRSS2 | −266 | −3.32 | −5.68 |
| 4. | KLK3 | −1.24 | −2.42 | −5.40 |
| 5. | NCAPD3 | −4.32 | −4.40 | −5.03 |
| 6. | NKX3-1 | −1.72 | −2.28 | −4.54 |
| 7. | NDRG1 | −2.04 | −4.17 | −4.37 |
| 8. | STEAP4 | −4.22 | −4.22 | −4.22 |
| 9. | FAM105A | −3.37 | −2.78 | −4.18 |
| 10. | AKAP12 | −1.91 | −3.84 | −4.10 |
| 11. | PMEPA1 | −3.05 | −2.16 | −4.05 |
| 12. | PLPP1 | −2.48 | −3.53 | −3.97 |
| 13. | SNAl2 | −3.97 | −1.79 | −3.97 |
| 14. | ACSL3 | −3.43 | −3.62 | −3.90 |
| 15. | ERRFl1 | −4.51 | −2.76 | −3.90 |
| 16. | CDC6 | −1.20 | −3.60 | −3.86 |
| 17. | ELL2 | −3.39 | −3.05 | −3.81 |
| 18. | CENPN | −3.31 | −2.13 | −3.79 |
| 19. | RHOU | −3.94 | −3.03 | −3.78 |
| 20. | EAF2 | −3.32 | −3.81 | −3.52 |

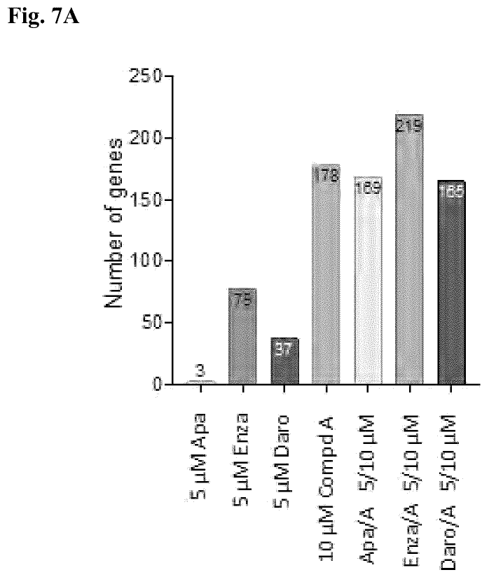

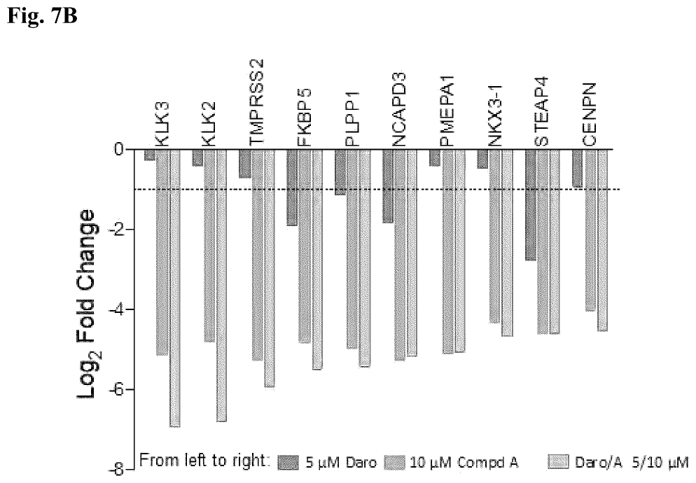

Apalutamide (Apa) or darolutamide (Daro) with or without Compound A was also assessed for gene expression in LNCaP cells. The number of genes significantly down regulated with Fold changes ≥4 is shown in FIG. 7A. Top ten down regulated genes with Daro/Compound A 5 μM/10 μM combination is shown in FIG. 7B. Darolutamide is not very active in LNCaP.

Data shown in FIGS. 6A-6C and 7A-7B and Table 4 were generated using NanoString Androgen Receptor/Prostate Cancer gene panel.

### Example 6. Inhibition of Androgen Responsive Genes in LNCaP95 Cells

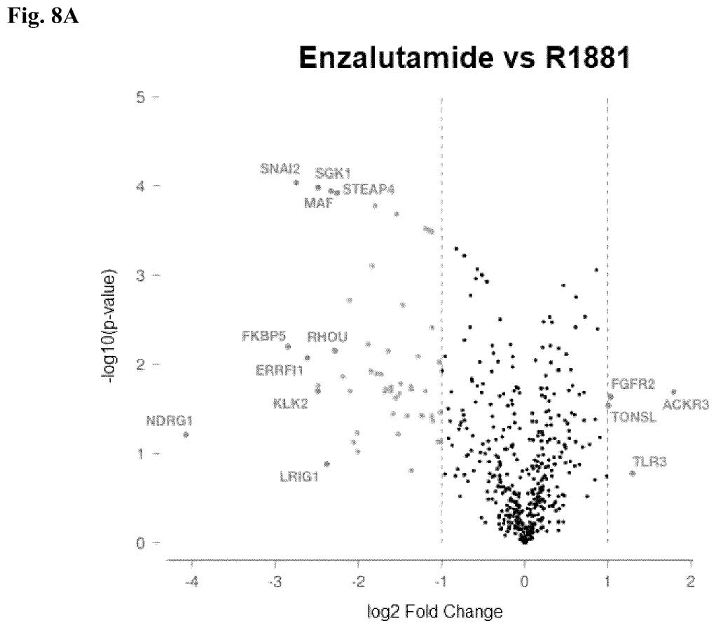

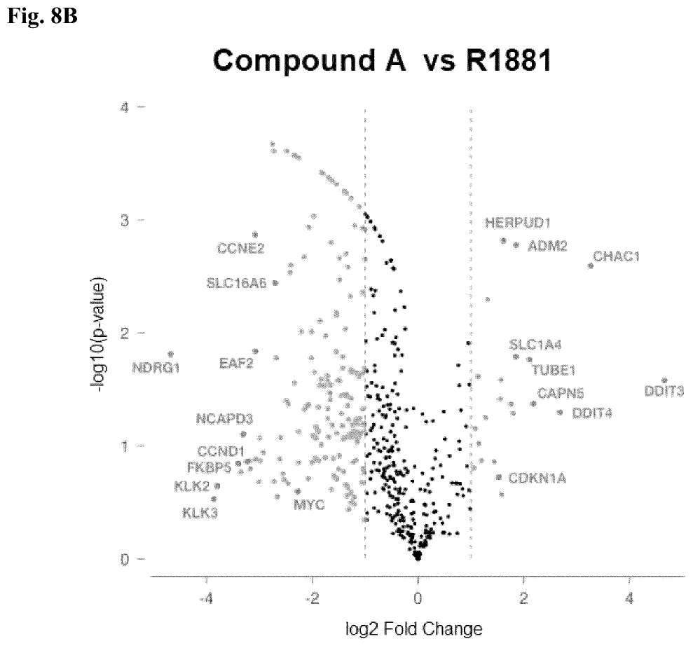

Relative mRNA expression in LNCaP95 (+R1881) was measured to assess response to Compound A alone or with enzalutamide (Enza). FIGS. 8A and 8B show log-ratio of gene expression values in LNCaP95 (+R1881) cells treated with enzalutamide compared to synthetic androgen (R1881) and LNCaP95 (+R1881) cells treated with Compound A compared to R1881, respectively.

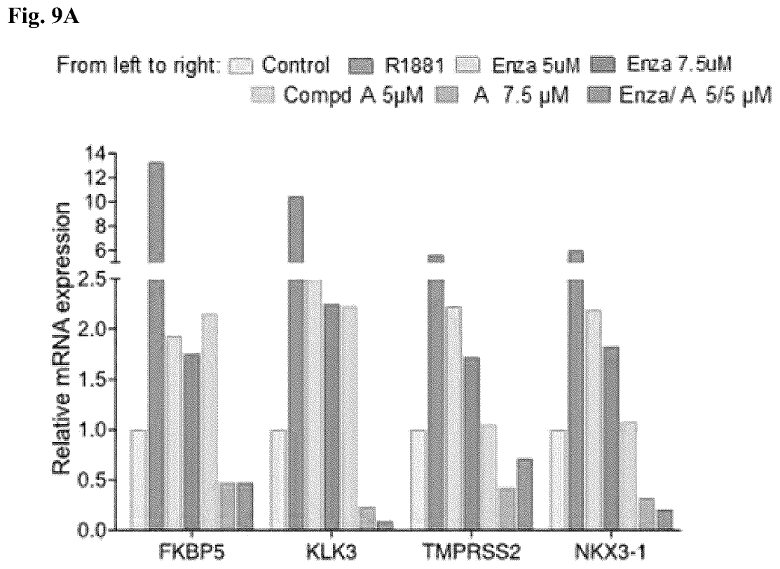

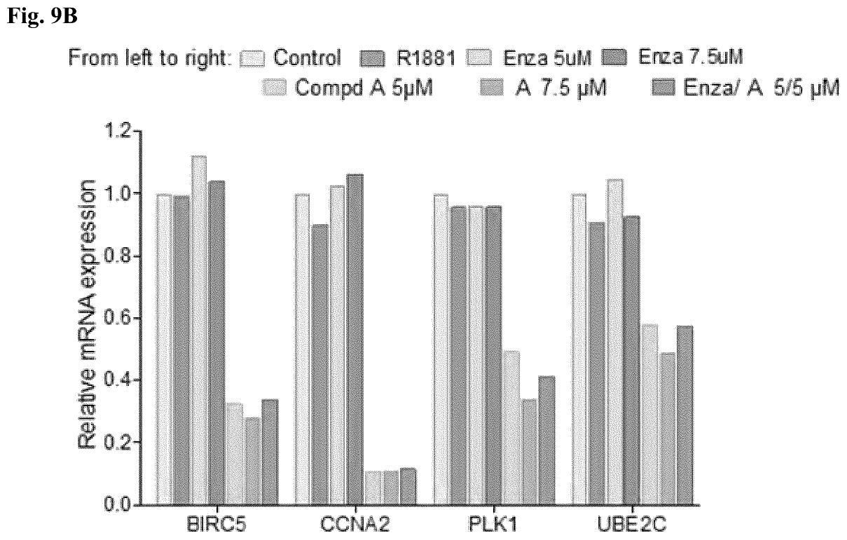

Relative mRNA expression in LNCaP95 (+R1881) was measured for Canonical AR target genes (FIG. 9A) and AR V7-activated genes (FIG. 9B). FIGS. 8A-8B and 9A-9B demonstrates that Compound A can modulate AR full-length and AR V7 driven genes. Table 5 shows top ten down-regulated genes with log 2 Fold change.

TABLE 5
Top 10 down-regulated genes (log2
Fold Change)—LNCaP95 (+R1881)

| | Enza 7.5 μM | | Compound A 7.5 μM |
|---|---|---|---|
| NDRG1 | −4.57 | KLK3 | −5.48 |
| FKBP5 | −2.92 | KLK2 | −5.07 |
| SNAI2 | −2.75 | NDRG1 | −4.47 |
| ERRFI1 | −2.69 | NKX3-1 | −4.23 |
| KLK2 | −2.61 | FKBP5 | −4.18 |
| SGK1 | −2.48 | CCND1 | −3.96 |
| SLC16A6 | −2.35 | ADAMTS1 | −3.96 |
| RHOU | −2.35 | PMEPA1 | −3.93 |
| TIPARP | −2.35 | PRR15L | −3.89 |
| IGF1R | −2.34 | NCAPD3 | −3.8 |

Data shown in FIGS. 8A-8B and 9A-9B and Table 5 were generated using NanoString Androgen Receptor/Prostate Cancer gene panel.

### Example 7: Inhibition of Androgen Responsive Genes in VCaP Cells

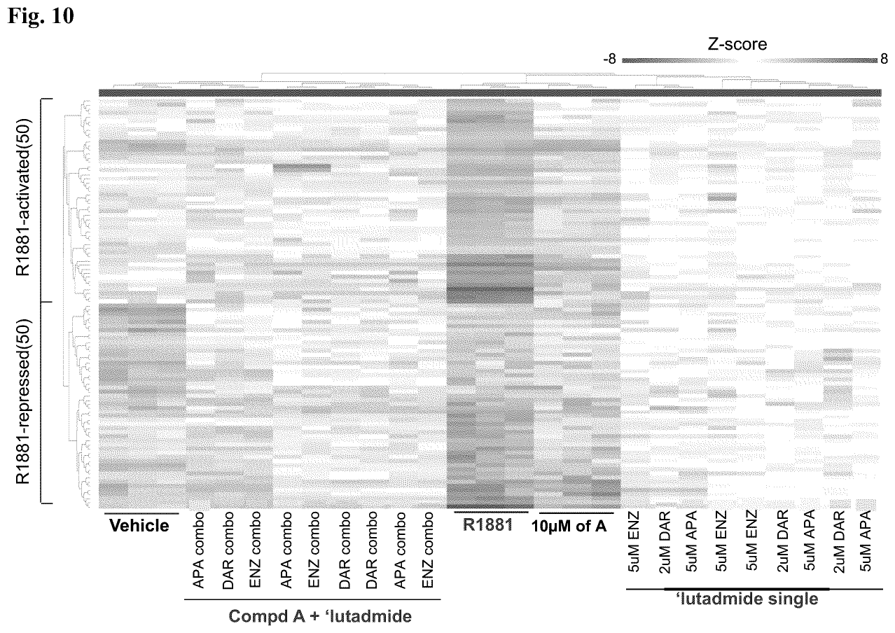

Relative mRNA expression in VCaP was measured to assess response of Compound A, enzalutamide (Enza), apalutamide (Apa), or darolutamide (Daro) alone or Compound A in combination with Enza, Apa, or Daro, in androgen responsive genes. FIG. 10 shows a heatmap from RNAseq analysis showing normalized expression of top 50 up- or down-regulated genes in response to R1881 stimulation (FC>2, padj<0.01). FIG. 10 demonstrates that Compound A at 10 μM can synergize with Enza, Apa, and Daro (collectively, “′lutamides”) and induces deeper androgen responsive gene expression in VCaP cells.

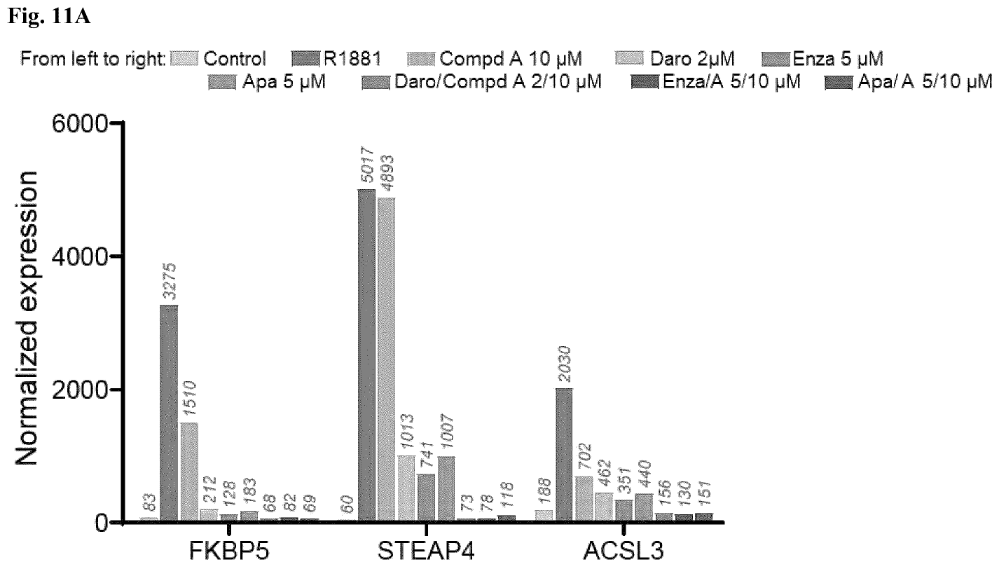

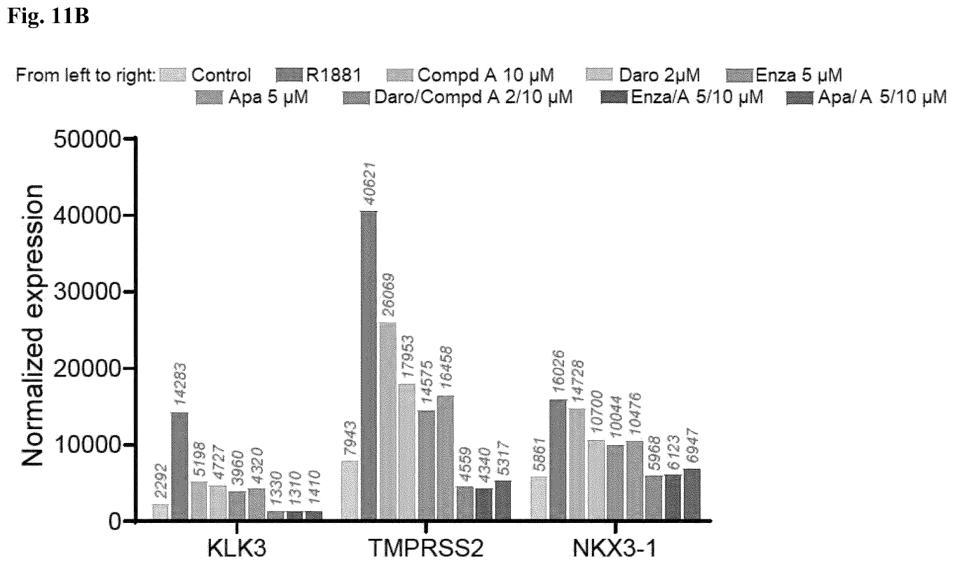

Further, the activity of Compound A at 10 μM in combination with the ′lutamides against canonical AR dependent genes showed superior inhibition of responsive genes as shown in FIGS. 11A and 11B.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1.-102. (canceled)

103. A method for modulating androgen receptor activity, comprising administering a pharmaceutical combination comprising Compound A having the structure: 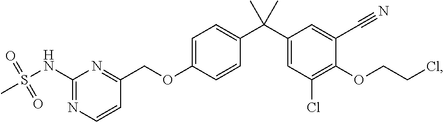 [SMILES: CC(C)(c1ccc(OCc2ccnc(NS(C)(=O)=O)n2)cc1)c1cc(Cl)c(OCCCl)c(C#N)c1]
or a pharmaceutically acceptable salt, solvate, stereoisomer or prodrug thereof and darolutamide to a subject in need thereof, wherein Compound A, or a pharmaceutically acceptable salt, solvate, stereoisomer or prodrug thereof, is administered at a daily dosage amount of about 50 mg and about 1500 mg.

104. The method of claim 103, wherein the modulating androgen receptor activity is for treating a condition or disease selected from prostate cancer, breast cancer, ovarian cancer, bladder cancer, pancreatic cancer, hepatocellular cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

105. The method of claim 103, wherein the modulating androgen receptor activity is for treating cancer.

106. (canceled)

107. The method of claim 105, wherein the cancer is prostate cancer.

108. The method of claim 107, wherein the prostate cancer is primary or localized prostate cancer, locally advanced prostate cancer, recurrent prostate cancer, advanced prostate cancer, metastatic prostate cancer, metastatic castration-resistant prostate cancer, or hormone-sensitive prostate cancer.

109. (canceled)

110. The method of claim 107, wherein the prostate cancer expresses full-length androgen receptor or truncated androgen receptor splice variant.

111. The method of claim 107, wherein the prostate cancer is resistant to enzalutamide monotherapy.

112.-113. (canceled)

114. The method of claim 103, wherein:
a) the combination of Compound A, or a pharmaceutically acceptable salt, solvate, stereoisomer or prodrug thereof, and darolutamide is in a single fixed dosage form; or
b) the combination of Compound A, or a pharmaceutically acceptable salt, solvate, stereoisomer or prodrug thereof, and darolutamide is in at least two dosage forms.

115. The method of claim 114, wherein the at least two dosage forms are co-packaged together into a single kit.

116. The method of claim 114, wherein at least one of the dosage forms comprises a solid dispersion formulation.

117. The method of claim 116, wherein the solid dispersion formulation is formed by solvent evaporation, hot-melt extrusion or spray drying dispersion.

118. The method of claim 116, wherein the solid dispersion formulation comprises one or more polymers selected from the group consisting of polyethylene glycol (PEG), polyvinyl pyrrolidone (PVP), polyethyleneoxide (PEO), poly(vinyl pyrrolidone-co-vinyl acetate) (PVP-VA), polymethacrylate, polyoxyethylene alkyl ether, polyoxyethylene-polyoxypropylene block copolymer, polyoxyethylene castor oil, polycaprolactam, polylactic acid, polyglycolic acid, poly(lactic-glycolic)acid, lipid, cellulose, pullulan, dextran, dextran acetate, dextran propionate, dextran succinate, dextran acetate propionate, dextran acetate succinate, dextran propionate succinate, dextran acetate propionate succinate, maltodextrin, hyaluronic acid, polysialic acid, chondroitin sulfate, heparin, fucoidan, pentosan polysulfate, spirulan, hydroxymethyl ethylcellulose, hydroxypropyl methylcellulose (HPMC), methyl cellulose, ethyl cellulose, hydroxyethyl cellulose, hydroxypropyl cellulose, carboxymethyl cellulose, carboxymethyl ethylcellulose (CMEC), sodium carboxymethyl cellulose, cellulose acetate succinate (CAS), methyl cellulose acetate succinate (MCAS), hydroxypropyl methylcellulose acetate succinate (HPMCAS), hydroxypropyl methylcellulose propionate succinate, hydroxypropyl methylcellulose propionate phthalate, cellulose acetate phthalate (CAP), hydroxypropyl methyl cellulose phthalate (HPMCP), hydroxypropyl methylcellulose acetate phthalate (HPMCAP), cellulose acetate trimellitate (CAT), hydroxypropyl methylcellulose acetate trimellitate (HPMCAT), hydroxypropyl methylcellulose propionate trimellitate, methyl cellulose acetate phthalate, hydroxypropyl cellulose acetate phthalate, cellulose acetate terephthalate, cellulose acetate isophthalate, cellulose acetate, cellulose butyrate, cellulose acetate butyrate, starch derivatives, dextran polymer derivative, poly(methacrylic acid-co-methyl methacrylate) 1:1, poly(methacrylic acid-co-methyl methacrylate) 1:2, poly(methacrylic acid-co-ethyl acrylate) 1:1, and a graft copolymers comprised of polyethylene glycol, polyvinyl caprolactam and polyvinyl acetate, or any combinations thereof.

119. The method of claim 117, wherein Compound A, or a pharmaceutically acceptable salt, solvate, stereoisomer or prodrug thereof, and darolutamide are in the same dosage form, and in the same solid dispersion formulation.

120. The method of claim 117, wherein Compound A, or a pharmaceutically acceptable salt, solvate, stereoisomer or prodrug thereof, and darolutamide are in the same dosage form, but separate solid dispersion formulation.

121. The method of claim 120, wherein each solid dispersion formulation each comprises different polymers.

122. The method of claim 103, wherein an amount of Compound A, or a pharmaceutically acceptable salt, solvate, stereoisomer or prodrug thereof, per a dosage form is between about 5 mg and 1,000 mg.

123. The method of claim 122, wherein an amount of Compound A per dosage form is about 5 mg, about 10 mg, about 15 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg.

124. The method of claim 103, wherein a daily dosage amount of darolutamide is between about 200 mg and about 3000 mg.

125. The method of claim 103, wherein a daily dosage amount of darolutamide is between about 400 mg and about 1500 mg.

126. The method of claim 123, wherein the dosage form is a tablet or a capsule.

127. The method of claim 103, wherein the daily dosage amount of Compound A is between about 100 mg and about 1000 mg.
